SEQUENCE LISTING (6 sequences)

SEQ ID NO: 1 - protein, 996 aa, Artificial Sequence
  tFrSc6-HN Construct
MELPILKANAITTILAAVTFCFASSQNITEEFYQSTCSAVSKGYLSALRTGWYTSVITIELSNIKENKCNGTDAKVKLMKQELDKYKNAVTELQLLMQSTPAANNQARGSGSGRSLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNVGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGSTNDLIQSGVNTRLLTIQSHVQNYIPISLTQQMSDLRKFISEITIRNDNQEVLPQRITHDVGIKPLNPDDFWRCTSGLPSLMKTPKIRLMPGPGLLAMPTTVDGCVRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSIPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAILNRTLSAGYTTTSCITHYNKGYCFHIVEINHKSLNTFQPMLFKTEIPKSCSGSGSGHHHHHHHHHHHH

SEQ ID NO: 2 - protein, 2991 aa, Artificial Sequence
  tFrSc6-HN Construct
ATGGAGCTGCCTATCCTGAAGGCCAACGCCATCACCACAATTCTGGCCGCCGTGACCTTCTGTTTTGCCAGCAGCCAGAACATCACCGAGGAGTTCTACCAGAGCACCTGTAGCGCCGTGAGCAAGGGCTATCTGAGCGCCCTGAGAACCGGCTGGTACACCAGCGTGATCACCATCGAGCTGAGCAACATCAAGGAGAACAAGTGCAACGGCACCGACGCCAAGGTGAAGCTGATGAAGCAGGAGCTGGACAAGTACAAGAACGCCGTGACCGAACTGCAGCTGCTGATGCAGTCTACCCCTGCCGCCAACAACCAGGCGAGAGGCAGCGGCTCTGGCAGAAGCCTGGGCTTTCTGCTGGGAGTGGGCTCTGCCATCGCCTCTGGCATCGCCGTGTCTAAGGTGCTGCACCTGGAGGGAGAGGTGAACAAGATCAAGAGCGCCCTGCTGAGCACCAATAAGGCCGTGGTGAGCCTGAGCAATGGCGTGAGCGTGCTGACAAGCAAGGTGCTGGACCTCAAGAACTACATCGACAAGCAGCTGCTGCCCATCGTGAACAAGCAGAGCTGCCGGATCAGCAACATCGAGACCGTGATCGAGTTCCAGCAGAAGAACAACCGGCTGCTGGAGATCACCAGGGAGTTCAGCGTGAATGTGGGCGTGACCACCCCTGTGAGCACCTACATGCTGACCAACAGCGAGCTGCTGAGCCTGATCAACGACATGCCCATCACCAACGACCAGAAGAAGCTGATGTCCAACAACGTGCAGATCGTGCGGCAGCAGAGCTACAGCATCATGTCCATCATCAAGGAGGAGGTGCTGGCTTACGTGGTGCAGCTGCCTCTGTACGGCGTGATCGACACCCCTTGCTGGAAGCTGCACACCAGCCCTCTGTGCACCACCAATACCAAGGAGGGCAGCAACATCTGCCTGACCAGGACCGATAGAGGCTGGTACTGCGACAATGCCGGCAGCGTGAGCTTCTTTCCACAGGCCGAGACCTGTAAGGTGCAGAGCAACCGGGTGTTCTGCGACACCATGAACAGCCTGACCCTGCCTTCTGAGGTGAACCTGTGCAACGTGGACATCTTCAACCCCAAGTACGACTGCAAGATCATGACCAGCAAGACCGACGTGAGCAGCAGCGTGATTACAAGCCTGGGCGCCATCGTGAGCTGTTACGGCAAGACCAAGTGCACCGCCAGCAACAAGAACCGCGGCATCATCAAGACCTTCAGCAACGGCTGCGACTACGTGAGCAACAAGGGCGTGGATACAGTGAGCGTGGGCAACACCCTGTACTACGTCAACAAGCAGGAGGGCAAGAGCCTGTACGTGAAGGGCGAGCCCATCATCAACTTCTACGACCCCCTGGTGTTCCCTAGCGACGAGTTCGATGCCAGCATCAGCCAGGTGAACGAGAAGATCAACCAGAGCCTGGCCTTCATCAGGAAGAGCGACGAGCTGCTGGGATCCACTAACGACCTGATCCAGTCTGGCGTGAACACAAGGCTGCTGACTATTCAGAGCCACGTCCAGAATTACATCCCAATTTCCCTGACACAGCAGATGTCTGACCTGAGGAAGTTCATCTCCGAAATCACTATTCGCAATGATAACCAGGAGGTGCTGCCCCAGCGCATCACCCATGACGTGGGCATCAAGCCACTGAACCCCGACGATTTTTGGAGGTGCACTTCAGGACTGCCTAGCCTGATGAAGACCCCAAAAATCCGACTGATGCCAGGACCTGGACTGCTGGCAATGCCAACCACAGTGGATGGATGCGTCCGAACCCCCTCTCTGGTCATCAACGACCTGATCTACGCCTATACTAGTAATCTGATCACCCGCGGCTGTCAGGACATTGGGAAGTCCTACCAGGTGCTGCAGATCGGCATCATTACAGTGAACAGTGATCTGGTCCCCGACCTGAATCCTCGCATCTCACACACTTTTAATATCAACGATAACCGAAAGTCATGCAGCCTGGCTCTGCTGAACACAGACGTGTACCAGCTGTGCTCTACTCCTAAAGTCGATGAACGGAGTGACTATGCAAGCTCCGGCATCGAGGATATTGTGCTGGACATCGTCAATTACGATGGGTCCATTTCTACTACCAGATTCAAGAACAATAACATCAGCTTTGACCAGCCCTACGCCGCTCTGTATCCATCCGTGGGACCAGGAATCTACTACAAGGGAAAAATCATTTTCCTGGGCTATGGCGGGCTGGAACACCCTATCAACGAGAATGTGATTTGCAACACAACTGGCTGTCCAGGGAAGACCCAGAGGGATTGCAATCAGGCCAGTCATTCACCCTGGTTTAGTGATCGGAGAATGGTGAACTCAATCATTGTGGTCGACAAAGGGCTGAATAGCATCCCTAAGCTGAAAGTCTGGACCATTTCAATGCGACAGAACTACTGGGGAAGCGAAGGCCGGCTGCTGCTGCTGGGCAATAAGATCTACATCTACACTCGGAGCACCTCCTGGCACTCCAAACTGCAGCTGGGGATCATTGACATCACCGATTATTCTGACATCCGGATTAAGTGGACATGGCACAACGTGCTGTCAAGACCCGGGAATAACGAGTGTCCTTGGGGACATAGCTGCCCAGATGGGTGTATCACCGGAGTGTACACAGACGCTTATCCTCTGAACCCAACCGGCAGTATCGTGTCTAGTGTCATTCTGGACTCTCAGAAAAGTAGAGTGAATCCCGTCATCACATACAGCACCGCAACAGAAAGAGTGAACGAGCTGGCCATTCTGAATAGGACTCTGAGCGCCGGATATACCACAACTTCCTGCATCACCCATTACAACAAGGGCTATTGTTTCCACATCGTGGAAATTAACCATAAAAGCCTGAATACCTTCCAGCCCATGCTGTTTAAGACAGAGATTCCTAAAAGTTGTTCAGGCAGTGGGTCAGGACATCACCATCATCATCATCATCACCATCATCACCATTGA

SEQ ID NO: 3 - protein, 996 aa, Artificial Sequence
  tFrSc8-HN Construct
MELPILKANAITTILAAVTFCFASSQNITEEFYQSTCSAVSKGYLSALRTGWYTSVITIELSNIKENKCNGTDAKVKLMKQELDKYKNAVTELQLLMQSTPAANNRARGSGSGRTLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNVGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGSTNDLIQSGVNTRLLTIQSHVQNYIPISLTQQMSDLRKFISEITIRNDNQEVLPQRITHDVGIKPLNPDDFWRCTSGLPSLMKTPKIRLMPGPGLLAMPTTVDGCVRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSIPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAILNRTLSAGYTTTSCITHYNKGYCFHIVEINHKSLNTFQPMLFKTEIPKSCSGSGSGHHHHHHHHHHHH

SEQ ID NO: 4 - protein, 2991 aa, Artificial Sequence
  tFrSc8-HN Construct
ATGGAGCTGCCTATCCTGAAGGCCAACGCCATCACCACAATTCTGGCCGCCGTGACCTTCTGTTTTGCCAGCAGCCAGAACATCACCGAGGAGTTCTACCAGAGCACCTGTAGCGCCGTGAGCAAGGGCTATCTGAGCGCCCTGAGAACCGGCTGGTACACCAGCGTGATCACCATCGAGCTGAGCAACATCAAGGAGAACAAGTGCAACGGCACCGACGCCAAGGTGAAGCTGATGAAGCAGGAGCTGGACAAGTACAAGAACGCCGTGACCGAACTGCAGCTGCTGATGCAGTCTACCCCTGCCGCCAACAACAGAGCCAGAGGCAGCGGCAGCGGCAGAACCCTGGGCTTTCTGCTGGGAGTGGGCTCTGCCATCGCCTCTGGCATCGCCGTGTCTAAGGTGCTGCACCTGGAGGGAGAGGTGAACAAGATCAAGAGCGCCCTGCTGAGCACCAATAAGGCCGTGGTGAGCCTGAGCAATGGCGTGAGCGTGCTGACAAGCAAGGTGCTGGACCTCAAGAACTACATCGACAAGCAGCTGCTGCCCATCGTGAACAAGCAGAGCTGCCGGATCAGCAACATCGAGACCGTGATCGAGTTCCAGCAGAAGAACAACCGGCTGCTGGAGATCACCAGGGAGTTCAGCGTGAATGTGGGCGTGACCACCCCTGTGAGCACCTACATGCTGACCAACAGCGAGCTGCTGAGCCTGATCAACGACATGCCCATCACCAACGACCAGAAGAAGCTGATGTCCAACAACGTGCAGATCGTGCGGCAGCAGAGCTACAGCATCATGTCCATCATCAAGGAGGAGGTGCTGGCTTACGTGGTGCAGCTGCCTCTGTACGGCGTGATCGACACCCCTTGCTGGAAGCTGCACACCAGCCCTCTGTGCACCACCAATACCAAGGAGGGCAGCAACATCTGCCTGACCAGGACCGATAGAGGCTGGTACTGCGACAATGCCGGCAGCGTGAGCTTCTTTCCACAGGCCGAGACCTGTAAGGTGCAGAGCAACCGGGTGTTCTGCGACACCATGAACAGCCTGACCCTGCCTTCTGAGGTGAACCTGTGCAACGTGGACATCTTCAACCCCAAGTACGACTGCAAGATCATGACCAGCAAGACCGACGTGAGCAGCAGCGTGATTACAAGCCTGGGCGCCATCGTGAGCTGTTACGGCAAGACCAAGTGCACCGCCAGCAACAAGAACCGCGGCATCATCAAGACCTTCAGCAACGGCTGCGACTACGTGAGCAACAAGGGCGTGGATACAGTGAGCGTGGGCAACACCCTGTACTACGTCAACAAGCAGGAGGGCAAGAGCCTGTACGTGAAGGGCGAGCCCATCATCAACTTCTACGACCCCCTGGTGTTCCCTAGCGACGAGTTCGATGCCAGCATCAGCCAGGTGAACGAGAAGATCAACCAGAGCCTGGCCTTCATCAGGAAGAGCGACGAGCTGCTGGGATCCACTAACGACCTGATCCAGTCTGGCGTGAACACAAGGCTGCTGACTATTCAGAGCCACGTCCAGAATTACATCCCAATTTCCCTGACACAGCAGATGTCTGACCTGAGGAAGTTCATCTCCGAAATCACTATTCGCAATGATAACCAGGAGGTGCTGCCCCAGCGCATCACCCATGACGTGGGCATCAAGCCACTGAACCCCGACGATTTTTGGAGGTGCACTTCAGGACTGCCTAGCCTGATGAAGACCCCAAAAATCCGACTGATGCCAGGACCTGGACTGCTGGCAATGCCAACCACAGTGGATGGATGCGTCCGAACCCCCTCTCTGGTCATCAACGACCTGATCTACGCCTATACTAGTAATCTGATCACCCGCGGCTGTCAGGACATTGGGAAGTCCTACCAGGTGCTGCAGATCGGCATCATTACAGTGAACAGTGATCTGGTCCCCGACCTGAATCCTCGCATCTCACACACTTTTAATATCAACGATAACCGAAAGTCATGCAGCCTGGCTCTGCTGAACACAGACGTGTACCAGCTGTGCTCTACTCCTAAAGTCGATGAACGGAGTGACTATGCAAGCTCCGGCATCGAGGATATTGTGCTGGACATCGTCAATTACGATGGGTCCATTTCTACTACCAGATTCAAGAACAATAACATCAGCTTTGACCAGCCCTACGCCGCTCTGTATCCATCCGTGGGACCAGGAATCTACTACAAGGGAAAAATCATTTTCCTGGGCTATGGCGGGCTGGAACACCCTATCAACGAGAATGTGATTTGCAACACAACTGGCTGTCCAGGGAAGACCCAGAGGGATTGCAATCAGGCCAGTCATTCACCCTGGTTTAGTGATCGGAGAATGGTGAACTCAATCATTGTGGTCGACAAAGGGCTGAATAGCATCCCTAAGCTGAAAGTCTGGACCATTTCAATGCGACAGAACTACTGGGGAAGCGAAGGCCGGCTGCTGCTGCTGGGCAATAAGATCTACATCTACACTCGGAGCACCTCCTGGCACTCCAAACTGCAGCTGGGGATCATTGACATCACCGATTATTCTGACATCCGGATTAAGTGGACATGGCACAACGTGCTGTCAAGACCCGGGAATAACGAGTGTCCTTGGGGACATAGCTGCCCAGATGGGTGTATCACCGGAGTGTACACAGACGCTTATCCTCTGAACCCAACCGGCAGTATCGTGTCTAGTGTCATTCTGGACTCTCAGAAAAGTAGAGTGAATCCCGTCATCACATACAGCACCGCAACAGAAAGAGTGAACGAGCTGGCCATTCTGAATAGGACTCTGAGCGCCGGATATACCACAACTTCCTGCATCACCCATTACAACAAGGGCTATTGTTTCCACATCGTGGAAATTAACCATAAAAGCCTGAATACCTTCCAGCCCATGCTGTTTAAGACAGAGATTCCTAAAAGTTGTTCAGGCAGTGGGTCAGGACATCACCATCATCATCATCATCACCATCATCACCATTGA

SEQ ID NO: 5 - protein, 996 aa, Artificial Sequence
  tFrSc10-HN Construct
MELPILKANAITTILAAVTFCFASSQNITEEFYQSTCSAVSKGYLSALRTGWYTSVITIELSNIKENKCNGTDAKVKLMKQELDKYKNAVTELQLLMQSTPAANNNARGSGSGRTLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNVGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGSTNDLIQSGVNTRLLTIQSHVQNYIPISLTQQMSDLRKFISEITIRNDNQEVLPQRITHDVGIKPLNPDDFWRCTSGLPSLMKTPKIRLMPGPGLLAMPTTVDGCVRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSIPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAILNRTLSAGYTTTSCITHYNKGYCFHIVEINHKSLNTFQPMLFKTEIPKSCSGSGSGHHHHHHHHHHHH

SEQ ID NO: 6 - protein, 2991 aa, Artificial Sequence
  tFrSc10-HN Construct
ATGGAGCTGCCTATCCTGAAGGCCAACGCCATCACCACAATTCTGGCCGCCGTGACCTTCTGTTTTGCCAGCAGCCAGAACATCACCGAGGAGTTCTACCAGAGCACCTGTAGCGCCGTGAGCAAGGGCTATCTGAGCGCCCTGAGAACCGGCTGGTACACCAGCGTGATCACCATCGAGCTGAGCAACATCAAGGAGAACAAGTGCAACGGCACCGACGCCAAGGTGAAGCTGATGAAGCAGGAGCTGGACAAGTACAAGAACGCCGTGACCGAACTGCAGCTGCTGATGCAGTCTACCCCTGCCGCCAACAACAACGCCAGAGGCAGCGGCAGCGGCAGAACCCTGGGCTTTCTGCTGGGAGTGGGCTCTGCCATCGCCTCTGGCATCGCCGTGTCTAAGGTGCTGCACCTGGAGGGAGAGGTGAACAAGATCAAGAGCGCCCTGCTGAGCACCAATAAGGCCGTGGTGAGCCTGAGCAATGGCGTGAGCGTGCTGACAAGCAAGGTGCTGGACCTCAAGAACTACATCGACAAGCAGCTGCTGCCCATCGTGAACAAGCAGAGCTGCCGGATCAGCAACATCGAGACCGTGATCGAGTTCCAGCAGAAGAACAACCGGCTGCTGGAGATCACCAGGGAGTTCAGCGTGAATGTGGGCGTGACCACCCCTGTGAGCACCTACATGCTGACCAACAGCGAGCTGCTGAGCCTGATCAACGACATGCCCATCACCAACGACCAGAAGAAGCTGATGTCCAACAACGTGCAGATCGTGCGGCAGCAGAGCTACAGCATCATGTCCATCATCAAGGAGGAGGTGCTGGCTTACGTGGTGCAGCTGCCTCTGTACGGCGTGATCGACACCCCTTGCTGGAAGCTGCACACCAGCCCTCTGTGCACCACCAATACCAAGGAGGGCAGCAACATCTGCCTGACCAGGACCGATAGAGGCTGGTACTGCGACAATGCCGGCAGCGTGAGCTTCTTTCCACAGGCCGAGACCTGTAAGGTGCAGAGCAACCGGGTGTTCTGCGACACCATGAACAGCCTGACCCTGCCTTCTGAGGTGAACCTGTGCAACGTGGACATCTTCAACCCCAAGTACGACTGCAAGATCATGACCAGCAAGACCGACGTGAGCAGCAGCGTGATTACAAGCCTGGGCGCCATCGTGAGCTGTTACGGCAAGACCAAGTGCACCGCCAGCAACAAGAACCGCGGCATCATCAAGACCTTCAGCAACGGCTGCGACTACGTGAGCAACAAGGGCGTGGATACAGTGAGCGTGGGCAACACCCTGTACTACGTCAACAAGCAGGAGGGCAAGAGCCTGTACGTGAAGGGCGAGCCCATCATCAACTTCTACGACCCCCTGGTGTTCCCTAGCGACGAGTTCGATGCCAGCATCAGCCAGGTGAACGAGAAGATCAACCAGAGCCTGGCCTTCATCAGGAAGAGCGACGAGCTGCTGGGATCCACTAACGACCTGATCCAGTCTGGCGTGAACACAAGGCTGCTGACTATTCAGAGCCACGTCCAGAATTACATCCCAATTTCCCTGACACAGCAGATGTCTGACCTGAGGAAGTTCATCTCCGAAATCACTATTCGCAATGATAACCAGGAGGTGCTGCCCCAGCGCATCACCCATGACGTGGGCATCAAGCCACTGAACCCCGACGATTTTTGGAGGTGCACTTCAGGACTGCCTAGCCTGATGAAGACCCCAAAAATCCGACTGATGCCAGGACCTGGACTGCTGGCAATGCCAACCACAGTGGATGGATGCGTCCGAACCCCCTCTCTGGTCATCAACGACCTGATCTACGCCTATACTAGTAATCTGATCACCCGCGGCTGTCAGGACATTGGGAAGTCCTACCAGGTGCTGCAGATCGGCATCATTACAGTGAACAGTGATCTGGTCCCCGACCTGAATCCTCGCATCTCACACACTTTTAATATCAACGATAACCGAAAGTCATGCAGCCTGGCTCTGCTGAACACAGACGTGTACCAGCTGTGCTCTACTCCTAAAGTCGATGAACGGAGTGACTATGCAAGCTCCGGCATCGAGGATATTGTGCTGGACATCGTCAATTACGATGGGTCCATTTCTACTACCAGATTCAAGAACAATAACATCAGCTTTGACCAGCCCTACGCCGCTCTGTATCCATCCGTGGGACCAGGAATCTACTACAAGGGAAAAATCATTTTCCTGGGCTATGGCGGGCTGGAACACCCTATCAACGAGAATGTGATTTGCAACACAACTGGCTGTCCAGGGAAGACCCAGAGGGATTGCAATCAGGCCAGTCATTCACCCTGGTTTAGTGATCGGAGAATGGTGAACTCAATCATTGTGGTCGACAAAGGGCTGAATAGCATCCCTAAGCTGAAAGTCTGGACCATTTCAATGCGACAGAACTACTGGGGAAGCGAAGGCCGGCTGCTGCTGCTGGGCAATAAGATCTACATCTACACTCGGAGCACCTCCTGGCACTCCAAACTGCAGCTGGGGATCATTGACATCACCGATTATTCTGACATCCGGATTAAGTGGACATGGCACAACGTGCTGTCAAGACCCGGGAATAACGAGTGTCCTTGGGGACATAGCTGCCCAGATGGGTGTATCACCGGAGTGTACACAGACGCTTATCCTCTGAACCCAACCGGCAGTATCGTGTCTAGTGTCATTCTGGACTCTCAGAAAAGTAGAGTGAATCCCGTCATCACATACAGCACCGCAACAGAAAGAGTGAACGAGCTGGCCATTCTGAATAGGACTCTGAGCGCCGGATATACCACAACTTCCTGCATCACCCATTACAACAAGGGCTATTGTTTCCACATCGTGGAAATTAACCATAAAAGCCTGAATACCTTCCAGCCCATGCTGTTTAAGACAGAGATTCCTAAAAGTTGTTCAGGCAGTGGGTCAGGACATCACCATCATCATCATCATCACCATCATCACCATTGA

(19) United States
(12) Patent Application Publication
(10) Pub. No.: US 2024/0197865 A1
(43) Pub. Date: Jun. 20, 2024

(54) COMPOSITIONS AND METHODS FOR PREVENTING RSV AND PIV3 INFECTIONS

(71) Applicant: University of Saskatchewan, Saskatoon (CA)

(72) Inventors: Sylvia Van Den Hurk, Saskatoon (CA); Volker Gerdts, Saskatoon (CA); Robert Brownlie, Saskatoon (CA); Ravendra Garg, Saskatoon (CA)

(21) Appl. No.: 18/556,668

(22) PCT Filed: Apr. 22, 2022
(86) PCT No.: PCT/CA2022/050621

§ 371 Date: Oct. 21, 2023

Related U.S. Application Data
(60) Provisional application No. 63/178,287, filed on Apr. 22, 2021.

(51) Int. Cl. A61K 39/295; A61K 39/00; A61K 39/39; A61P 37/04; C07K 14/005

(52) U.S. Cl. CPC A61K 39/295; A61K 39/39; A61P 37/04; C07K 14/005; A61K 2039/55511; A61K 2039/55516; A61K 2039/55555; A61K 2039/55561; A61K 2039/6018; A61K 2039/70; C12N 2760/18522; C12N 2760/18534; C12N 2760/18622; C12N 2760/18634

(57) ABSTRACT

The present invention provides immunogenic chimeric respiratory syncytial virus (RSV)-parainfluenza virus type 3 (PIV3) compounds and associated compositions, along with methods of treating, preventing and/or diagnosing RSV- and PIV3-related disorders.

### TECHNICAL FIELD

The present invention relates generally to viral pathogens. In particular, the invention pertains to respiratory syncytial virus (RSV)- and parainfluenza virus type 3 (PIV3)-immunogenic compositions and methods of treating, preventing and/or diagnosing RSV- and PIV3-related disorders.

### BACKGROUND

Human respiratory syncytial virus (RSV) and parainfluenza virus type 3 (PIV3) are the most common causative agents of acute lower respiratory tract infections in infants and children. RSV is an Orthopneumovirus within the family Pneumoviridae. RSV is highly infectious; >99% of children have been infected at least once by 2 years of age. The clinical presentation of RSV resembles the common cold in healthy older children and adults, which manifests itself as rhinitis, sore throat and cough. However, RSV causes much more serious disease in infants and the elderly (1, 2). In infants, RSV is the most common cause of broncheolitis and pneumonia. While antigenically quite stable, RSV elicits a short-term immune response, but fails to induce a long-term protective response leaving individuals susceptible to re-infection throughout adolescence and adulthood. In the elderly RSV can cause pneumonia and exacerbation of COPD (3). RSV infects the nasopharyngeal epithelium and may spread to the bronchiolar epithelium leading to necrosis, lymphocytic peribronchiolar infiltration and submucosal edema. Mucus, sloughed epithelium and lymphoid aggregates then obstruct the bronchioles.

Human parainfluenzaviruses (hPIVs) within the Paramyxoviridae family cause upper and lower respiratory tract infections (4) and are the second most common cause of lower respiratory disease in young children after RSV. While reports vary about the exact numbers, hPIVs account for 17-18% of all viruses isolated from pediatric patients (5, 6). Human parainfluenzaviruses primarily attack epithelial cells in the respiratory tract. Common disease symptoms caused by hPIVs include rhinorrhea (runny nose), cough, croup (acute laryngotracheobronchitis), bronchiolitis, and pneumonia, and in some cases high body temperatures of up to ˜40° C. (1, 6, 2). There are still no effective antiviral treatments for either RSV or PIV3. Both RSV and PIV3 are enveloped viruses with a single-stranded, non-segmented, negative-sense RNA. On the basis of antigenic and genetic analysis, PIV has been divided into four subtypes, PIV-1 to -4. PIV3 is the most pathogenic form of PIV and is associated with significant morbidity and mortality among infants and young children (5, 7). RSV encodes two major surface glycoproteins, namely the attachment (G) and fusion (F) proteins, whereas PIV3 encodes hemagglutinin-neuraminidase (HN) and F proteins. The RSV G and PIV3 HN proteins initiate viral infection in host cells by binding to receptors on the plasma membrane. The RSV and PIV3 F proteins facilitate penetration by fusion of the viral envelope with the plasma membrane of the host cell. Both glycoproteins play a critical role in pathogenesis of these viruses and are major targets for neutralizing antibodies (7, 8, 9), making them subunit vaccine candidates.

Several vaccine candidates against RSV and PIV3 have been developed and evaluated in rodents and humans, but no licensed human vaccines are yet available to prevent these infections (7, 10, 11). Vaccination with purified PIV3 F or HN protein alone is not sufficient to induce full protective immunity due to poor immunogenicity in animals and humans (12, 13).

Recently, maternal vaccination has been proposed as an approach to induce high levels of pathogen-specific neutralizing antibodies in pregnant women. These antibodies are transferred to the offspring and provide effective short-term protection to young infants during a period of susceptibility to pathogens, such as Bordetella pertussis, Clostridium tetani and influenza virus (14). Maternally derived antibodies have also been proven to be transferred passively and promote protection against respiratory viruses such as RSV (15). Another example is a clinical trial with maternal vaccination against tetanus, diphtheria, and acellular pertussis that showed higher levels of antibodies early in infancy (16).

### SUMMARY OF THE INVENTION

The inventors herein have successfully designed and characterized chimeric RSV-PIV3 antigens for use in subunit vaccine compositions.

Accordingly, the present invention provides RSV-PIV3 subunit compositions for the treatment and/or prevention of RSV and/or PIV3 infections, such as pneumonia or bronchiolitis, in humans. Subunit vaccines, including immunogens and mixtures of immunogens derived from RSV and PIV3 isolates, are used to provide protection against subsequent infection. The present invention thus provides a commercially useful method of preventing and/or treating RSV and/or PIV3 infections in humans.

In an embodiment, there is provided an immunogenic fusion protein comprising a polypeptide with at least 90% sequence identity to a polypeptide selected from the group consisting of:
a polypeptide comprising amino acid residues 26 to 979 of SEQ ID NO: 1,
a polypeptide comprising amino acid residues 26 to 979 of SEQ ID NO: 3, and
a polypeptide comprising amino acid residues 26 to 979 of SEQ ID NO: 5.

In another embodiment, there is provided an immunogenic composition comprising:
a polypeptide with at least 90% sequence identity to a fusion polypeptide selected from the group consisting of:
   a polypeptide comprising amino acid residues 26 to 979 of SEQ ID NO: 1,
   a polypeptide comprising amino acid residues 26 to 979 of SEQ ID NO: 3, and
   a polypeptide comprising amino acid residues 26 to 979 of SEQ ID NO: 5,
and a pharmaceutically acceptable excipient.

In a further embodiment, the immunogenic composition further comprises an immunological adjuvant.

In yet a further embodiment, the adjuvant comprises: (a) a polyphosphazene; (b) a CpG oligonucleotide or a poly (I:C); and (c) a host defense peptide.

In an embodiment, the composition is for administration to a human subject.

In another embodiment, the adjuvant is formulated with a mucoadhesive lipidic carrier to produce a mucoadhesive lipidic carrier system.

In a further embodiment, the mucoadhesive lipidic carrier of the system comprises a cationic liposome.

In yet a further embodiment, the mucoadhesive lipid carrier comprises one or more cationic lipids selected from 1,2-dioleoyl-3-trimethylammonium-propane (DOTAP); 3β-[N-(N′,N′-dimethylaminoethane)-carbamoyl] (DC); dimethyldioctadecylammonium (DDA); octadecylamine (SA); dimethyldioctadecylammonium bromide (DDAB); 1,2-dioleoyl-sn-glycerol-3-phosphoethanolamine (DOPE); egg L-α-phosphatidylcholine (EPC); cholesterol (Chol); distearoylphosphatidylcholine (DSPC); 1,2-dimyristoyl-3-trimethylammonium-propane (DMTAP); dimyristoylphosphatidylcholine (DMPC); or ceramide carbamoyl-spermine (CCS).

In an embodiment, there is provided a method of treating or preventing RSV and/or PIV3 infection in a subject, comprising administering the fusion protein as described herein to said subject, such that said RSV and/or PIV3 infection is treated or prevented in said subject.

In another embodiment, there is provided a method of treating or preventing RSV and/or PIV3 infection in a subject, comprising administering the immunogenic composition as described herein to the subject, such that the RSV and/or PIV3 infection is treated or prevented in the subject.

In a particular embodiment, the fusion protein or immunogenic composition as described herein is administered to a human subject.

In another particular embodiment, the immunogenic composition as described herein is administered parenterally, intramuscularly, intravenously, intraperitoneally, subcutaneously, orally, intranasally, or as an aerosol.

In another embodiment, there is provided an immunogenic composition as described herein, wherein the polyphosphazene is one or more of: poly[di(sodium carboxylatophenoxy)phosphazene] (PCPP), poly(di-4-oxyphenylproprionate)phosphazene (PCEP), compound 37:

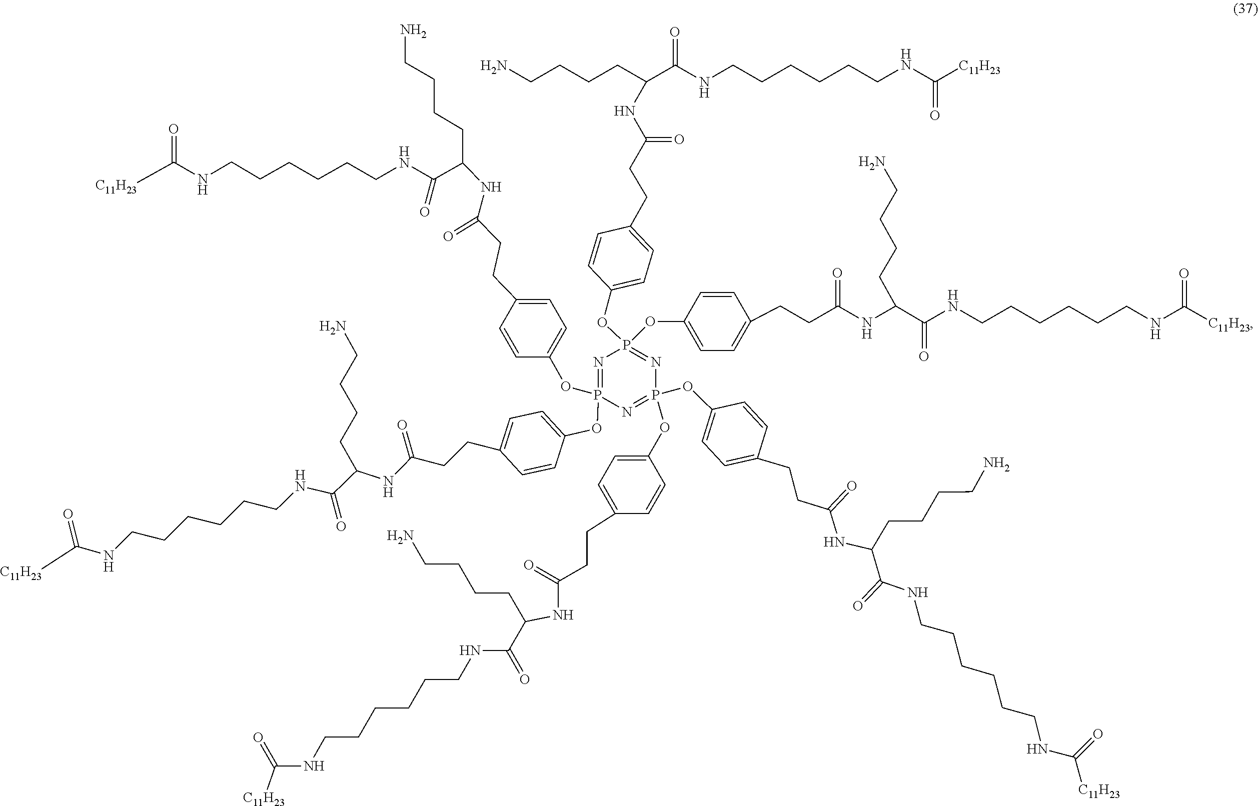

or compound 39:

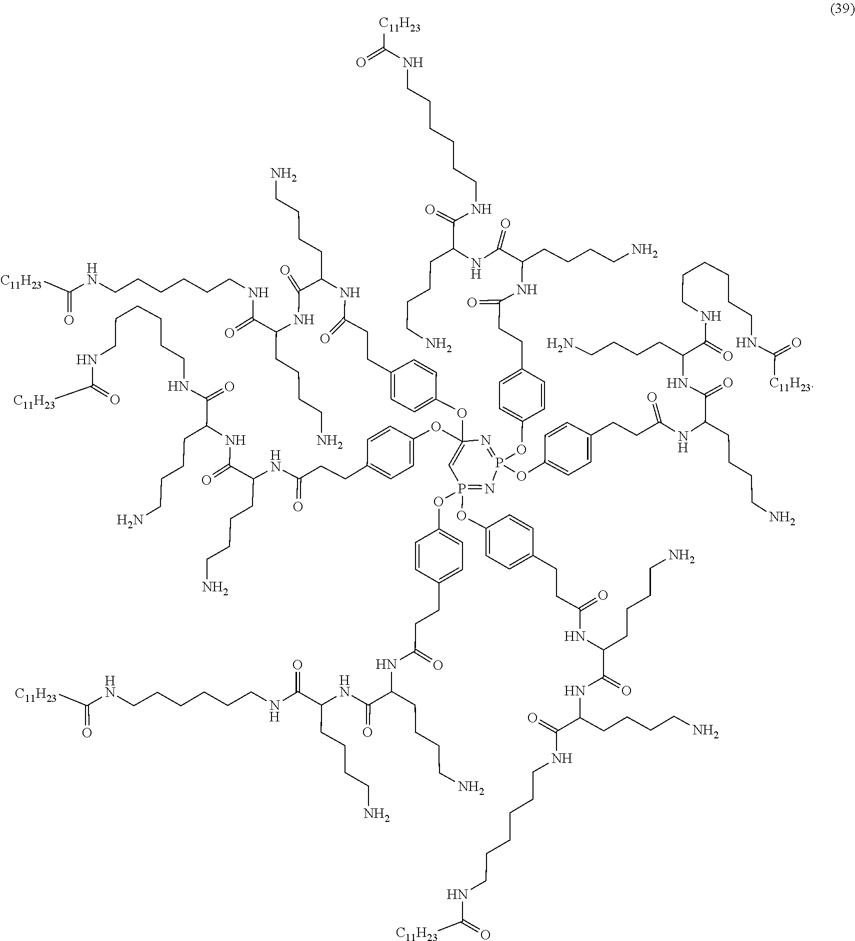

In a particular embodiment, the polyphosphazene is compound 37.

In another particular embodiment, the polyphosphazene is compound 39.

These and other embodiments of the subject invention will readily occur to those of skill in the art in view of the disclosure herein.

### BRIEF DESCRIPTION OF THE DRAWINGS

These and other features of the invention will become more apparent from the following description in which reference is made to the appended drawings wherein:

FIG. 1 shows purified recombinant protein tFrSc6-HN that was analysed by SDS-PAGE under non-reducing conditions. Lane 1, 5 μL; Lane 2, 2.5 μL. Arrows refer to the migration of markers 245, 190, 135, 100, 80, and 58 kDa.

FIG. 2 shows antibody titers as determined by ELISA on serum samples collected before RSV challenge in mouse Trial No.1. Groups of five mice were immunized twice with a mock vaccine (PBS) or one of three TriAdj-adjuvanted vaccines, tFrSc6-HN+PCEP-TriAdj, tFrSc6-HN+CPZ37-TriAdj and tFrSc6-HN+CPZ39-TriAdj. FIG. 2A shows IgG1 titers and FIG. 2B shows IgG2a titers. Individual values and median with interquartile range are shown. *P<0.05; **P<0.01.

FIG. 3 shows RSV (A) and PIV3 (B)-neutralizing antibody titers determined in sera collected at necropsy in mouse Trial No.1. Virus neutralization titers (“VN titers”) are expressed as the highest dilution of serum that resulted in <50% of cells displaying cytopathic effects. Individual values and median with interquartile range are shown. *P<0.05; **P<0.01.

FIG. 4 shows IFN-γ (A)-secreting and IL-5 (B) -secreting T cells per million splenocytes from mouse Trial 1. Cytokine-secreting cell numbers are expressed as the difference in the number of spots between tFrSc6-HN-stimulated wells and medium-control wells. Individual values and median with interquartile range are shown. *P<0.05; **P<0.01.

FIG. 5 shows virus replication in the lungs determined after RSV challenge in mouse Trial No.1. Virus replication in the lungs is expressed as PFU per gram of lung tissue. Individual values and median with interquartile range are shown.

FIG. 6 shows antibody titers determined by ELISA on serum samples collected four days after RSV challenge in mouse Trial No.2. Groups of five mice were immunized twice with a mock vaccine (PBS), one of four TriAdj-adjuvanted vaccines, tFrSc6-HN+PCEP-TriAdj, tFrSc6-HN+CPZ37-TriAdj and tFrSc6-HN+CPZ39-TriAdj, or with tFrSc6-HN+aluminum hydroxide (“alum”). FIG. 5A shows IgG1 titers and FIG. 5B shows IgG2a titers. Individual values and median with interquartile range are shown. *P<0.05; **P<0.01.

FIG. 7 shows RSV (A) and PIV3 (B)-neutralizing antibody titers determined in sera collected at necropsy in mouse Trial No.2. Virus neutralization titers (“VN titers”) are expressed as the highest dilution of serum that resulted in <50% of cells displaying cytopathic effects. Individual values and median with interquartile range are shown. *P<0.05; **P<0.01.

FIG. 8 shows virus replication in the lungs determined after RSV challenge in mouse Trial No.2. Virus replication in the lungs is expressed as PFU per gram of lung tissue. Individual values and median with interquartile range are shown.

FIG. 9 shows production of IFN-γ (A) and IL-5 (B) in the lung homogenates collected at necropsy in mouse Trial No.2. Individual values and median with interquartile range are shown.

FIG. 10 shows antibody titers determined by ELISA on serum samples collected four days after RSV challenge in cotton rat Trial No.1. Groups of six cotton rats were immunized twice with a mock vaccine (PBS), one of four TriAdj-adjuvanted vaccines, tFrSc6-HN+PCEP-TriAdj, tFrSc6-HN+CPZ37-TriAdj and tFrSc6-HN+A+B-TriAdj, or with tFrSc6-HN+aluminum hydroxide (“alum”). Individual values and median with interquartile range are shown.

FIG. 11 shows RSV (A) and PIV3 (B)-neutralizing antibody titers determined in sera collected at necropsy in cotton rat Trial No.1. Virus neutralization titers (“VN titers”) are expressed as the highest dilution of serum that resulted in <50% of cells displaying cytopathic effects. Individual values and median with interquartile range are shown.

FIG. 12 shows virus replication in the lungs (A) and nasal wash (B) determined after RSV challenge in cotton rat Trial No.3. Virus replication in the lungs is expressed as PFU per gram of lung tissue and PFU per ml of nasal wash. Individual values and median with interquartile range are shown.

### DETAILED DESCRIPTION OF THE INVENTION

The following description is of preferred embodiments by way of example only and without limitation to the combination of features necessary for carrying the invention into effect.

The practice of the present invention will employ, unless otherwise indicated, conventional methods of virology, chemistry, biochemistry, recombinant DNA techniques and immunology, within the skill of the art. Such techniques are explained fully in the literature. See, e.g., Fundamental Virology, Current Edition, vol. I & II (B. N. Fields and D. M. Knipe, eds.); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company); A. L. Lehninger, Biochemistry (Worth Publishers, Inc., current edition); Sambrook, et al., Molecular Cloning: A Laboratory Manual (current edition); Methods In Enzymology (S. Colowick and N. Kaplan eds., Academic Press, Inc.).

All publications, patents and patent applications cited herein, whether supra or infra, are hereby incorporated by reference in their entireties.

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proline: Pro (P) | Serine: Ser (S) |
| Threonine: Thr (T) | Tryptophan: Trp (W) |
| Tyrosine: Tyr (Y) | Valine: Val (V) |

### 1. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an antigen” includes a mixture of two or more such antigens, and the like.

RSV is an Orthopneumovirus within the family Pneumoviridae and causes a number of disorders, described further herein. RSV infects the nasopharyngeal epithelium and then can spread to the bronchiolar epithelium. This leads to necrosis, and subsequent lymphocytic peribronchiolar infiltration and submucosal edema. RSV infects the nasopharyngeal epithelium and may spread to the bronchiolar epithelium leading to necrosis, lymphocytic peribronchiolar infiltration and submucosal edema. Ultimately, widespread mucus plugging, increased expiratory resistance and partial airway obstruction lead to wheezing, atelectasis and hyperinflation.

RSV is highly infectious and accounts for >60% of acute lower respiratory tract infections (LRTI) in children and >80% in infants <1 year of age; this makes RSV the most common cause of pediatric broncheolitis and pneumonia (2). Almost all children have been infected at least once, and nearly half of those twice, by the age of 2 (17). The clinical presentation of RSV resembles the common cold in healthy older children and adults, which manifests itself as rhinitis, sore throat and cough. However, RSV causes much more serious disease in infants and the elderly (1, 2). In infants, RSV is the most common cause of broncheolitis and pneumonia. Disease caused by RSV is specifically severe in children between 2 and 3 months of age, when maternal antibodies (MatAbs) start to decline (18). Morbidity and mortality are most prevalent in pre-mature infants and infants with chronic lung or congenital heart disease (19). Worldwide, 1:200 infants are hospitalized annually for LRTI, with a mortality rate of ˜5%, which amounts to more than 1 million deaths (19, 20). An increased incidence of asthma has been associated with more severe LRTI. While antigenically quite stable, RSV elicits a short-term immune response, but fails to induce a long-term protective response leaving individuals susceptible to re-infection throughout adolescence and adulthood (3). In the elderly RSV can cause pneumonia and exacerbation of COPD (3).

The term RSV intends any subspecies, strain or isolate of the organism which is capable of causing disease.

PIV3 is a Respirovirus within the family Paramyxoviridae and causes a number of associated disorders, described further herein. The term PIV3 intends any subspecies, strain or isolate of the organism which is capable of causing disease. Human parainfluenzaviruses are the second most common cause of lower respiratory tract infections in young children after respiratory syncytial virus (RSV) (4). While reports vary about the exact numbers, PIVs account for 17-18% of all viruses isolated from pediatric patients (5, 6). Human parainfluenzaviruses primarily attack epithelial cells in the respiratory tract. Common disease symptoms caused by PIVs include rhinorrhea (runny nose), cough, croup (acute laryngotracheobronchitis), bronchiolitis, and pneumonia, and in some cases high body temperatures of up to ˜40° C. (4). PIVs belong to the Paramyxoviridae, with two genera: Respirovirus (PIV 1 and 3) and Rubulavirus (PIV 2 and 4). Bronchiolitis and pneumonia are caused by all four types of PIVs, but more cases have been associated with hPIV 1 and 3. During a 20-year study, PIV1, PIV2 and PIV3 were responsible for, respectively, 6.0, 3.2 and 11% of hospitalizations of infants and children (21). PIVs have also been linked to acute and chronic neurological diseases, including febrile seizures, encephalitis, ventriculitis, and cluster headaches. Other conditions such as development of apnea and bradycardia due to hPIV infection have also been reported in rare cases in infants, and the majority of deaths have been in infants. PIV3 infections occur earliest and most frequently, like RSV, in infants <6 months old; in the USA 50% of 1-year old children and almost all 6-year-olds have been infected with PIV3.

The term PIV3 intends any subspecies, strain or isolate of the organism which is capable of causing disease.

The term “derived from” is used herein to identify the original source of a molecule but is not meant to limit the method by which the molecule is made which can be, for example, by chemical synthesis or recombinant means.

An “RSV molecule” is a molecule derived from the virus, including, without limitation, polypeptide, protein, antigen, polynucleotide, oligonucleotide, and nucleic acid molecules from any of the various RSV subspecies, strains, or isolates. A “PIV3 molecule” is a molecule derived from the virus, including, without limitation, polypeptide, protein, antigen, polynucleotide, oligonucleotide, and nucleic acid molecules from any of the various PIV3 subspecies, strains, or isolates. An “RSV-PIV3 molecule” is a chimeric (i.e. a fused) molecule comprising components derived from both RSV and PIV3, including, without limitation, polypeptide, antigen, polynucleotide, oligonucleotide and nucleic acid molecules from any of the various RSV and PIV3 subspecies, strains, or isolates. The molecule need not be physically derived from the particular viruses in question, but may be synthetically or recombinantly produced.

Nucleic acid and polypeptide sequences from a number of RSV and PIV3 isolates are known and/or described herein. Representative RSV-PIV3 proteins, and polynucleotides encoding the proteins, for use in treating and/or preventing RSV and/or PIV3 infection, are presented in Tables 2 and 3 herein. Additional representative sequences found in isolates from various mammals are reviewed in Mansi et al., 2019 (“A Contemporary View of Respiratory Syncytial Virus (RSV) Biology and Strain-Specific Differences”), and listed in the National Center for Biotechnology Information (NCBI) database (22).

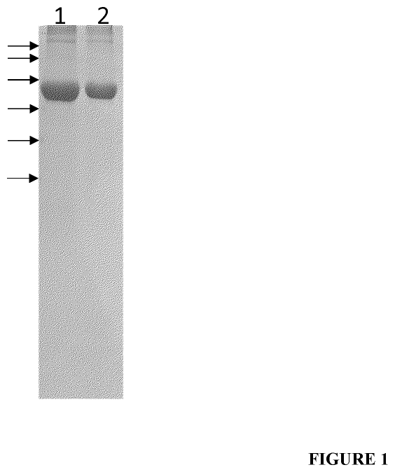

However, an “RSV-PIV3 molecule”, such as an antigen, as defined herein, is not limited to those shown and described in Tables 2, 3 and FIGS. 1-5, as various RSV and PIV3 isolates are known and variations in sequences may occur between them. Thus, an “RSV-PIV3 molecule” as defined herein intends a chimeric molecule comprising an F protein component from an RSV isolate or strain and an HN protein component from a PIV3 isolate or strain. Components from both RSV A strains and RSV B strains are contemplated (22).

By “RSV disease” or “RSV disorder” is meant a disease or disorder caused in whole or in part by an RSV infection. As explained herein, RSV invades the respiratory tract to lead to disease pathogenesis. Thus, the term intends both clinical and subclinical disease. Clinical symptoms of RSV include rhinorrhea (runny nose), sore throat, cough, croup (acute laryngotracheobronchitis), fever, bronchiolitis, and pneumonia, and in very young infants sometimes dyspnea, cyanosis, tachypnea, tachycardia, and/or respiratory wheeze.

By “PIV3 disease” or “PIV3 disorder” is meant a disease or disorder caused in whole or in part by a PIV3 infection. As explained herein, PIV3 invades the respiratory tract to lead to disease pathogenesis. Thus, the term intends both clinical and subclinical disease. While adults generally get mild upper respiratory infection, parainfluenza viruses may cause croup, bronchitis, pharyngitis, and pneumonia in children. Symptoms of parainfluenza pneumonia may include fever, cough, coryza, dyspnea, crackles, or wheezes. The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions, to the native sequence, so long as the protein maintains the desired activity. These modifications may be deliberate, as through site-directed mutagenesis, or may be accidental, such as through mutations of hosts which produce the proteins or errors due to PCR amplification.

The term “peptide” as used herein refers to a fragment of a polypeptide. Thus, a peptide can include a C-terminal deletion, an N-terminal deletion and/or an internal deletion of the native polypeptide, so long as the entire protein sequence is not present. A peptide will generally include at least about 3-10 contiguous amino acid residues of the full-length molecule, and can include at least about 15-25 contiguous amino acid residues of the full-length molecule, or at least about 20-50 or more contiguous amino acid residues of the full-length molecule, or any integer between 3 amino acids and the number of amino acids in the full-length sequence, provided that the peptide in question retains the ability to elicit the desired biological response.

By “immunogenic” protein, polypeptide or peptide is meant a molecule which includes one or more epitopes and thus can modulate an immune response. Such peptides can be identified using any number of epitope mapping techniques, well known in the art. See, e.g., Epitope Mapping Protocols in Methods in Molecular Biology (2018) (Johan Rockberg and Johan Nilvebrant, Eds.) Springer, New York. For example, linear epitopes may be determined by for example, software programs, (See., e.g., Saha et al., Structure, Function, and Bioinformatics (2006) 65:40-48); or by concurrently synthesizing large numbers of peptides on solid supports, the peptides corresponding to portions of the protein molecule, and reacting the peptides with antibodies while the peptides are still attached to the supports. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots.

Immunogenic molecules, for the purposes of the present invention, will usually be at least about 5 amino acids in length, such as, for example, about 10 to about 15 or more amino acids in length. There is no critical upper limit to the length of the molecule, which can comprise the full-length of the protein sequence, or even a fusion protein comprising two or more epitopes, proteins, antigens, etc.

As used herein, the term “epitope” generally refers to the site on an antigen which is recognized by a T-cell receptor and/or an antibody. Several different epitopes may be carried by a single antigenic molecule. The term “epitope” also includes modified sequences of amino acids which stimulate responses against the whole organism. The epitope can be generated from knowledge of the amino acid and corresponding DNA sequences of the polypeptide, as well as from the nature of particular amino acids (e.g., size, charge, etc.) and the codon dictionary, without undue experimentation. See, e.g., Ivan Roitt, Essential Immunology; Janis Kuby, Immunology.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytotoxic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity, of nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

Thus, an immunological response as used herein may be one that stimulates the production of antibodies. The antigen of interest may also elicit production of CTLs. Hence, an immunological response may include one or more of the following effects: the production of antibodies by B-cells; and/or the activation of suppressor T-cells and/or memory/effector T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art, such as, but not limited to, those described in the Examples herein.

The innate immune system of mammals also recognizes and responds to molecular features of pathogenic organisms via activation of Toll-like receptors and similar receptor molecules on immune cells. Upon activation of the innate immune system, various non-adaptive immune response cells are activated to, e.g., produce various cytokines, lymphokines and chemokines. Cells activated by an innate immune response include immature and mature dendritic cells of the monocyte and plasmacytoid lineage (MDC, PDC), as well as gamma, delta, alpha and beta T cells and B cells and the like. Thus, the present invention also contemplates an immune response wherein the immune response involves both an innate and adaptive response.

An “immunogenic composition” is a composition that comprises an immunogenic molecule where administration of the composition to a subject results in the development in the subject of a humoral and/or a cellular immune response to the molecule of interest.

An “antigen” refers to a molecule, such as a protein, polypeptide, or fragment thereof, containing one or more epitopes (either linear, conformational or both) that will stimulate a host's immune-system to make a humoral and/or cellular antigen-specific response. The term is used interchangeably with the term “immunogen.” Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide which expresses an antigen or antigenic determinant in vivo, such as in DNA immunization applications, is also included in the definition of antigen herein.

By “subunit vaccine” is meant a vaccine composition that includes one or more selected antigens but not all antigens, derived from or homologous to, an antigen from a pathogen of interest. Such a composition is substantially free of intact pathogen cells or pathogenic particles, or the lysate of such cells or particles. Thus, a “subunit vaccine” can be prepared from at least partially purified (preferably substantially purified) immunogenic molecules from the pathogen, or analogs thereof. The method of obtaining an antigen included in the subunit vaccine can thus include standard purification techniques, recombinant production, or synthetic production.

“Substantially purified” generally refers to isolation of a substance such that the substance comprises the majority percent of the sample in which it resides. Typically in a sample, a substantially purified component comprises at least 50%, preferably at least 80%-85%, and even more preferably at least 90% of the sample. Techniques for purifying molecules of interest are well-known in the art and include, for example, ion-exchange chromatography, affinity chromatography and sedimentation according to density.

By “isolated” is meant that the indicated molecule is separate and discrete from the whole organism with which the molecule is found in nature or is present in the substantial absence of other biological macromolecules of the same type.

An “antibody” intends a molecule that “recognizes,” i.e., specifically binds to an epitope of interest present in an antigen. By “specifically binds” is meant that the antibody interacts with the epitope in a “lock and key” type of interaction to form a complex between the antigen and antibody, as opposed to non-specific binding that might occur between the antibody and, for instance, components in a mixture that includes the test substance with which the antibody is reacted. The term “antibody” as used herein includes antibodies obtained from both polyclonal and monoclonal preparations, as well as, the following: hybrid (chimeric) antibody molecules; F(ab′)₂ and F(ab) fragments; Fv molecules (non-covalent heterodimers; single-chain Fv molecules (sFv); dimeric and trimeric antibody fragment constructs; minibodies; humanized antibody molecules; and any functional fragments obtained from such molecules, wherein such fragments retain immunological binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)₂, Fv, and other fragments, as well as chimeric and humanized homogeneous antibody populations, that exhibit immunological binding properties of the parent monoclonal antibody molecule.

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-99% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis. See, e.g., molbiol-tools.ca/alignments for a list of computer programs to determine similarity between two or more amino acid or nucleotide sequences. These programs are readily utilized with the default parameters recommended by the manufacturer. For example, percent identity of a particular nucleotide sequence to a reference sequence can be determined using the homology Smith-Waterman algorithm with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., Molecular Cloning, a laboratory manual, Cold Spring Harbor Laboratories, New York.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oregon, as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

“Recombinant host cells”, “host cells,” “cells”, “cell lines,” “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

The term “transform” is used to refer to the uptake of foreign DNA by a cell. A cell has been “transformed” when exogenous DNA has been introduced inside the cell membrane. A number of transformation techniques are generally known in the art. See, e.g., Sambrook et al., Molecular Cloning, a laboratory manual, Cold Spring Harbor Laboratories, New York; Davis et al. Basic Methods in Molecular Biology, Elsevier. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses. When used for immunization, such gene delivery expression vectors may be referred to as vaccines or vaccine vectors.

By “therapeutically effective amount” in the context of the immunogenic compositions described herein is meant an amount of an immunogen which will induce an immunological response as described herein, either for antibody production or for treatment or prevention of infection.

As used herein, “treatment” refers to, without limitation, any of (i) the prevention of infection or reinfection, as in a traditional vaccine, and/or (ii) the reduction or elimination of symptoms from an infected individual. Treatment may be effected prophylactically (prior to infection) or therapeutically (following infection). Additionally, prevention or treatment in the context of the present invention can be reduction of the amount of RSV and/or PIV3 virus present in the treated subject.

### 2. MODES OF CARRYING OUT THE INVENTION

Before describing the present invention in detail, it is to be understood that this invention is not limited to particular formulations or process parameters, and as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based in part on the discovery of immunogenic chimeric RSV-PIV3 molecules and their usefulness in subunit vaccines against RSV and PIV3. The RSV-PIV3 molecules and the compositions of the present invention are contemplated to provide protection against both RSV A and B and PIV3 strains. These molecules include one or more epitopes for stimulating an immune response in a subject of interest. The molecules are provided as fusion proteins. The chimeric antigens of the present invention can be incorporated into pharmaceutical compositions, such as vaccine compositions. As shown in the examples, serum from animals immunized with subunit vaccine compositions including RSV and PIV3 recombinant proteins contained virus neutralizing antibodies. Upon RSV challenge, no virus was detected in vaccinated mice, thus indicating these neutralizing antibodies prevented RSV.

The present invention thus provides immunological compositions and methods for treating and/or preventing RSV and PIV3 diseases. Immunization can be achieved by any of the methods known in the art including, but not limited to, use of vaccines containing RSV and PIV3 antigens or fusion proteins comprising multiple antigens, or by passive immunization using antibodies directed against the antigens. Such methods are described in detail below. Moreover, the antigens described herein can be used for detecting the presence of RSV- or PIV3-specific antibodies, for example in a biological sample.

In order to further an understanding of the invention, a more detailed discussion is provided below regarding chimeric RSV-PIV3 antigens, production thereof, compositions comprising the same, and methods of using such compositions in the treatment and/or prevention of infection, as well as in the diagnosis of infection.

### A. RSV and PIV3 Antigens

Antigenic components for use in the subject compositions can be derived from any of the several RSV and PIV3 strains and isolates.

Table 2 and Table 3 show representative chimeric RSV-PIV3 molecules for use in compositions for stimulating immune responses against RSV and PIV3. The molecules listed in Tables 2 and Table 3 were found to be immunogenic and protective as described in the Examples.

In a preferred embodiment, the compositions comprise at least one of the RSV-PIV3 antigens as listed in Tables 2 and 3. Moreover, it is also contemplated that the antigen(s) present in the composition(s) can include the full-length amino acid sequences of the aforementioned RSV-PIV3 antigens, or fragments or variants of these sequences, so long as the antigens stimulate an immunological response, preferably, a neutralizing and/or protective immune response. Thus, the antigens can be provided with deletions from the N- or C-termini which do not disrupt immunogenicity, including without limitation, deletions of an N-terminal methionine if present, deletions of all or part of the transmembrane domain(s) if present, deletions of all or part of the cytoplasmic domain(s) if present, and deletions of the native signal sequence if present. Additionally, the molecules can include other N-terminal, C-terminal and internal deletions of amino acids or sequences irrelevant to immunogenicity. Moreover, the molecules can include additions, such as the presence of a heterologous signal sequence if desired, as well as amino acid linkers, and/or ligands useful in protein purification, such as histidine tags, c-Myc tags, FLAG tags, V5 tags, HA tags, glutathione-S-transferase or staphylococcal protein A.

Representative fusion proteins are described in detail below and are shown in Table 2 and Table 3. It is to be understood that the present invention is not limited to the use of these representative proteins, as a number of strains and isolates of RSV and PIV3 are known, and the use of corresponding RSV F and PIV3 HN proteins from these strains and isolates in similar fusion proteins is contemplated and intended to be captured herein.

As explained above, any of the fusion proteins listed in Tables 2 and 3, as well as variants thereof, such as proteins with substantial sequence identity thereto, e.g., sequences that exhibit at least about 50% sequence identity, such as at least about 75% sequence identity, e.g., at least about 80% or 85% sequence identity, for example at least about 90% sequence identity, such as at least about 95% or 99% sequence identity or more, over a defined length of the molecules, or any integer within these values, will find use herein. Additionally, the corresponding RSV F and PIV3 HN antigens from different strains or isolates of RSV and/or PIV, can be used to create the fusion protein of the immunogenic compositions described herein, to provide protection against a broad range of RSV and PIV3 strains.

The antigen sequences present in the fusion proteins may be separated by spacers. A selected spacer sequence may encode a wide variety of moieties of one or more amino acids in length. Selected spacer groups may also provide enzyme cleavage sites so that the expressed chimera can be processed by proteolytic enzymes in vivo to yield a number of peptides.

For example, amino acids can be used as spacer sequences. Such spacers will typically include from 1-500 amino acids, such as 1-100 amino acids, e.g., 1-50 amino acids, such as 1-25 amino acids, 1-10 amino acids, 1-3, 1-4, 1-5, 1-6, amino acids, or any integer between 1-500. The spacer amino acids may be the same or different between the various antigens. Particularly preferred amino acids for use as spacers are amino acids with small side groups, such as serine, alanine, glycine and valine, various combinations of amino acids or repeats of the same amino acid. For example, linker sequences including a particular amino acid or combination of amino acids, such as glycine, or glycine-serine, etc. may include 2, 3, 4, 5, 6, 7, 8, 9, 10 . . . 20 . . . 25 . . . 30, etc. of such repeats.

Although particular fusions are exemplified herein which include spacer sequences, it is also to be understood that one or more of the antigens present in the fusion constructs can be directly adjacent to another antigen, without an intervening spacer sequence.

In order to enhance immunogenicity of the RSV-PIV3 fusions of multiple antigen molecules, they may be conjugated with a carrier. By “conjugated” is meant that the protein and fusions of interest may be linked to the carrier via non-covalent interactions, such as by electrostatic forces, or by covalent bonds, and the like. Thus, the carrier may be linked to the protein of interest via recombinant production, or the protein may be synthetically or chemically linked to a carrier after or during production. By “carrier” is meant any molecule which when associated with an antigen of interest, imparts immunogenicity to the antigen. Examples of suitable carriers include large, slowly metabolized macromolecules such as: proteins; polysaccharides, such as sepharose, agarose, cellulose, cellulose beads and the like; polymeric amino acids such as polyglutamic acid, polylysine, and the like; amino acid copolymers; inactive virus particles; bacterial toxins such as tetanus toxoid, serum albumins, keyhole limpet hemocyanin, thyroglobulin, ovalbumin, sperm whale myoglobin, and other proteins well known to those skilled in the art. Other suitable carriers for the antigens of the present invention may be used and will be known to the person of skill in the art.

These carriers may be used in their native form or their functional group content may be modified by, for example, succinylation of lysine residues or reaction with Cys-thiolactone. A sulfhydryl group may also be incorporated into the carrier (or antigen) by, for example, reaction of amino functions with 2-iminothiolane or the N-hydroxysuccinimide ester of 3-(4-dithiopyridyl propionate. Suitable carriers may also be modified to incorporate spacer arms (such as hexamethylene diamine or other bifunctional molecules of similar size) for attachment of peptides.

As explained above, carriers can be physically conjugated to the proteins of interest, using standard coupling reactions. Alternatively, chimeric molecules can be prepared recombinantly for use in the present invention, such as by fusing a gene encoding a suitable polypeptide carrier to one or more copies of a gene, or fragment thereof, encoding for selected RSV and PIV3 multiple antigen fusion molecules.

Preferably, the above described antigens and fusions, are produced recombinantly. A polynucleotide encoding these proteins can be introduced into an expression vector which can be expressed in a suitable expression system. A variety of bacterial, yeast, mammalian and insect expression systems are available in the art and any such expression system can be used. Optionally, a polynucleotide encoding these proteins can be translated in a cell free translation system. Such methods are well known in the art. The proteins also can be constructed by solid phase protein synthesis.

It is also contemplated that the fusion proteins of the present invention, or the individual components of these proteins, may or may not contain other amino acid sequences, such as amino acid linkers or signal sequences, either native or heterologous, as well as ligands useful in protein purification, such as glutathione S transferase and staphylococcal protein A.

### B. RSV-PIV3 Polynucleotides

RSV-PIV3 polynucleotides encoding the chimeric RSV-PIV3 antigens for use in the subject compositions can be derived from any RSV and PIV3 strains or isolates.

Representative polynucleotide sequences encoding the RSV-PIV3 antigens are shown in Table 3. The polynucleotides can be modified for expression in a particular host cell, such as baculovirus.

The polynucleotide sequences encoding RSV-PIV3 chimeric antigens will encode the full-length amino acid sequences, or fragments or variants of these sequences so long as the resulting antigens stimulate an immunological response, preferably, a protective immune response. Thus, the polynucleotides can encode antigens with deletions or additions, as described above.

Once the coding sequences for the desired antigens have been isolated or synthesized, they can be cloned into any suitable vector or replicon for expression. Numerous cloning vectors are known to those of skill in the art, and the selection of an appropriate cloning vector is a matter of choice. A variety of bacterial, yeast, plant, mammalian and insect expression systems are available in the art and any such expression system can be used. Optionally, a polynucleotide encoding these proteins can be translated in a cell-free translation system. Such methods are well known in the art.

Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces), bovine papilloma virus (mammalian cells), pCMV (mammalian cells), pEB (mammalian cells) and EBNA-based Episomal vectors (mammalian cells). See, generally, Sambrook et al., Molecular Cloning, a laboratory manual, Cold Spring Harbor Laboratories, New York.

Insect cell expression systems, such as baculovirus systems, can also be used and are known to those of skill in the art. Plant expression systems can also be used to produce the immunogenic proteins. Generally, such systems use virus-based vectors to transfect plant cells with heterologous genes.

Viral systems, such as a vaccinia based infection/transfection system, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation product(s).

The coding sequence can be placed under the control of a promoter, ribosome binding site (for bacterial expression) and, optionally, an operator (collectively referred to herein as “control elements”), so that the DNA sequence encoding the desired antigen is transcribed into RNA in the host cell transformed by a vector containing this expression construction. The coding sequence may or may not contain a signal peptide or leader sequence. Leader sequences can be removed by the host in post-translational processing.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector which already contains the control sequences and an appropriate restriction site.

In some cases it may be necessary to modify the coding sequence so that it may be attached to the control sequences with the appropriate orientation; i.e., to maintain the proper reading frame. It may also be desirable to produce mutants or analogs of the immunogenic proteins. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., Molecular Cloning, a laboratory manual, Cold Spring Harbor Laboratories, New York.

The expression vector is then used to transform an appropriate host cell. A number of mammalian cell lines are known in the art and include immortalized cell lines available from the American Type Culture Collection (ATCC), such as, but not limited to, human embryonic kidney 293 (HEK293) cells, Chinese hamster ovary (CHO) cells, HeLa cells, baby hamster kidney (BHK) cells, monkey kidney cells (COS), human hepatocellular carcinoma cells (e.g., Hep G2), as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the proteins of the present invention are produced by growing host cells transformed by an expression vector described above under conditions whereby the protein of interest is expressed. The selection of the appropriate growth conditions is within the skill of the art. If the proteins are not secreted, the cells are then disrupted, using chemical, physical or mechanical means, which lyse the cells yet keep the proteins substantially intact. Following disruption of the cells, cellular debris is removed, generally by centrifugation. Whether produced intracellularly or secreted, the protein can be further purified, using standard purification techniques such as but not limited to, column chromatography, ion-exchange chromatography, size-exclusion chromatography, electrophoresis, high-performance liquid chromatography (HPLC), immunoadsorbent techniques, affinity chromatography, immunoprecipitation, and the like.

### C. Antibodies

The antigens of the present invention can be used to produce antibodies for therapeutic (e.g., passive immunization), diagnostic and purification purposes. These antibodies may be polyclonal or monoclonal antibody preparations, monospecific antisera, or may be hybrid or chimeric antibodies, such as humanized antibodies, altered antibodies, F(ab′)₂ fragments, F(ab) fragments, Fv fragments, single-domain antibodies, dimeric or trimeric antibody fragment constructs, minibodies, or functional fragments thereof which bind to the antigen in question. Antibodies are produced using techniques well known to those of skill in the art.

For subjects known to have an RSV-related disease, an anti-RSV-antigen antibody may have therapeutic benefit and can be used to confer passive immunity to the subject in question. For subjects known to have a PIV3-related disease, an anti-PIV3-antigen antibody may have therapeutic benefit and can be used to confer passive immunity to the subject in question. Alternatively, antibodies can be used in diagnostic applications, described further below, as well as for purification of the antigen of interest.

### D. Compositions

The RSV-PIV3 chimeric molecules can be formulated into compositions for delivery to subjects for eliciting an immune response, such as for inhibiting infections. Compositions of the invention may comprise or be co-administered with non-RSV or non-PIV3 antigens or with a combination of RSV and PIV3 antigens, as described above. Methods of preparing such formulations are described in, e.g., Remington's Pharmaceutical Sciences, Mack Publishing Company, Easton, Pennsylvania, 22nd Edition, 2012. The compositions of the present invention can be prepared as injectables, either as liquid solutions or suspensions. Solid forms suitable for solution in or suspension in liquid vehicles prior to injection may also be prepared. The preparation may also be emulsified or the active ingredient encapsulated in liposome vehicles. The active immunogenic ingredient is generally mixed with a compatible pharmaceutical vehicle, such as, for example, water, saline, dextrose, glycerol, ethanol, or the like, and combinations thereof. In addition, if desired, the vehicle may contain minor amounts of auxiliary substances such as wetting or emulsifying agents and pH buffering agents.

Adjuvants which enhance the effectiveness of the composition may also be added to the formulation. Such adjuvants include any compound or combination of compounds that act to increase an immune response to an RSV- or a PIV3-antigen or combination of antigens, thus reducing the quantity of antigen necessary in the vaccine, and/or the frequency of injection necessary in order to generate an adequate immune response.

For example, a triple adjuvant formulation as described in, e.g., U.S. Pat. No. 9,061,001, incorporated herein by reference in its entirety, can be used in the subject compositions. The triple adjuvant formulation includes a host defense peptide (“HDP”), in combination with a polyanionic polymer such as a polyphosphazene, and a nucleic acid sequence possessing immunostimulatory properties (ISS), such as an oligodeoxynucleotide molecule with or without a CpG motif (a cytosine followed by guanosine and linked by a phosphate bond) or the synthetic dsRNA analog poly(I:C).

Examples of host defense peptides for use in the combination adjuvant, as well as individually with the chimeric antigens of the present invention include, without limitation, HH2 (VQLRIRVAVIRA); IDR-1002 (VQRWLIVWRIRK); IDR-1018 (VRLIVAVRIWRR); Indolicidin (ILPWKWPWWPWRR); HH111 (ILKWKWPWWPWRR); HH113 (ILPWKKPWWPWRR); HH970 (ILKWKWPWWKWRR); HH1010 (ILRWKWRWWRWRR); Nisin Z (Ile-Dhb-Ala-Ile-Dha-Leu-Ala-Abu-Pro-Gly-Ala-Lys-Abu-Gly-Ala-Leu-Met-Gly-Ala-Asn-Met-Lys-Abu-Ala-Abu-Ala-Asn-Ala-Ser-Ile-Asn-Val-Dha-Lys); JK1 (VFLRRIRVIVIR); JK2 (VFWRRIRVWVIR); JK3 (VQLRAIRVRVIR); JK4 (VQLRRIRVWVIR); JK5 (VQWRAIRVRVIR); and JK6 (VQWRRIRVWVIR). Any of the above peptides, as well as fragments and analogs thereof, that display the appropriate biological activity, such as the ability to modulate an immune response, such as to enhance an immune response to a co-delivered antigen, will find use herein.

Exemplary, non-limiting examples of ISSs for use in the triple adjuvant composition, or individually include, CpG oligonucleotides or non-CpG molecules. By “CpG oligonucleotide” or “CpG ODN” is meant an immunostimulatory nucleic acid containing at least one cytosine-guanine dinucleotide sequence (i.e., a 5′ cytidine followed by 3′ guanosine and linked by a phosphate bond) and which activates the immune system. An “unmethylated CpG oligonucleotide” is a nucleic acid molecule which contains an unmethylated cytosine-guanine dinucleotide sequence (i.e., an unmethylated 5′ cytidine followed by 3′ guanosine and linked by a phosphate bond) and which activates the immune system. A “methylated CpG oligonucleotide” is a nucleic acid which contains a methylated cytosine-guanine dinucleotide sequence (i.e., a methylated 5′ cytidine followed by a 3′ guanosine and linked by a phosphate bond) and which activates the immune system. CpG oligonucleotides are well known in the art and described in, e.g., U.S. Pat. Nos. 6,194,388; 6,207,646; 6,214,806; 6,218,371; 6,239,116; and 6,339,068; PCT Publication No. WO 01/22990; PCT Publication No. WO 03/015711; US Publication No. 20030139364, which patents and publications are incorporated herein by reference in their entireties.

Examples of such CpG oligonucleotides include, without limitation, 5′TCCATGACGTTCCTGACGTT3′, termed CpG ODN 1826, a Class B CpG; 5′TCGTCGTTGTCGTTTTGTCGTT3′, termed CpG ODN 2007, a Class B CpG; 5′TCGTCGTTTTGTCGTTTTGTCGTT3′, also termed CpG 7909 or 10103, a Class B CpG; 5′ GGGGACGACGTCGTGGGGGGG3′, termed CpG 8954, a Class A CpG; and 5′TCGTCGTTTTCGGCGCGCGCCG3′, also termed CpG 2395 or CpG 10101, a Class C CpG. All of the foregoing class B and C molecules are fully phosphorothioated.

Non-CpG oligonucleotides for use in the present composition include the double stranded polyriboinosinic acid:polyribocytidylic acid, also termed poly(I:C); and a non-CpG oligonucleotide 5′AAAAAAGGTACCTAAATAGTATGTTTCTGAAA3′.

Polyanionic polymers for use in the triple combination adjuvants or alone include polyphosphazenes. Typically, polyphosphazenes for use with the present adjuvant compositions will either take the form of a polymer in aqueous solution or a polymer microparticle, with or without encapsulated or adsorbed substances such as antigens or other adjuvants. For example, the polyphosphazene can be a soluble polyphosphazene, such as a polyphosphazene polyelectrolyte with ionized or ionizable pendant groups that contain, for example, carboxylic acid, sulfonic acid or hydroxyl moieties, and pendant groups that are susceptible to hydrolysis under conditions of use to impart biodegradable properties to the polymer. Such polyphosphazene polyelectrolytes are well known and described in, for example, U.S. Pat. Nos. 5,494,673; 5,562,909; 5,855,895; 6,015,563; and 6,261,573, incorporated herein by reference in their entireties. Alternatively, polyphosphazene polymers in the form of cross-linked microparticles will also find use herein. Such cross-linked polyphosphazene polymer microparticles are well known in the art and described in, e.g., U.S. Pat. Nos. 5,053,451; 5,149,543; 5,308,701; 5,494,682; 5,529,777; 5,807,757; 5,985,354; and 6,207,171, incorporated herein by reference in their entireties.

It will be understood that by a person of skill in the art that “polyphosphazene” is a cyclic or acyclic (unless otherwise specified), high-molecular weight, water-soluble polymer, containing a backbone of alternating phosphorous and nitrogen atoms and organic side groups or ligands attached at each phosphorus atom. See, e.g., Payne et al., Vaccine (1998) 16:92-98; Andrianov & Payne, Adv. Drug. Deliv. Rev. (1998) 31:185-196. Examples of linear polyphosphazene polymers for use herein include poly[di(sodium carboxylatophenoxy)phosphazene] (PCPP) and poly(di-4-oxyphenylproprionate)phosphazene (PCEP), in various forms, such as the sodium salt, or acidic forms, as well as a polymer composed of varying percentages of PCPP or PCEP copolymer with hydroxyl groups, such as 90:10 PCPP/OH. Methods for synthesizing these compounds are known and described in the patents referenced above, as well as in Andrianov et al., Biomacromolecules (2004) 5:1999; Andrianov et al., Macromolecules (2004) 37:414; Mutwiri et al., Vaccine (2007) 25:1204. Examples of cyclic polyphosphazenes for use herein are described below, such as CPZ37 and CPZ39.

In another embodiment, the aforementioned triple adjuvant formulation may be further formulated with a mucoadhesive cationic lipidic carrier, to form a mucoadhesive lipidic carrier system, as described in WO/2020/056524, which is herein incorporated by reference in its entirety. In a preferred embodiment, antigens of the present invention or compositions thereof along with the mucoadhesive carrier and triple adjuvant are administered intramuscularly or mucosally.

Additional adjuvants include chitosan-based adjuvants, and any of the various saponins, oils, and other substances known in the art, such as AMPHIGEN™ which comprises de-oiled lecithin dissolved in an oil, usually light liquid paraffin. In vaccine preparations, AMPHIGEN™ is dispersed in an aqueous solution or suspension of the immunizing antigen as an oil-in-water emulsion. Other adjuvants are LPS, bacterial cell wall extracts, bacterial DNA, synthetic oligonucleotides and combinations thereof (Schijns et al., Curr. Opi. Immunol. (2000) 12:456), Mycobacterial phlei (M. phlei) cell wall extract (MCWE) (U.S. Pat. No. 4,744,984), M. phlei DNA (M-DNA) and M-DNA-M phlei cell wall complex (MCC). For example, compounds which may serve as emulsifiers herein include natural and synthetic emulsifying agents, as well as anionic, cationic and nonionic compounds. Among the synthetic compounds, anionic emulsifying agents include, for example, the potassium, sodium and ammonium salts of lauric and oleic acid, the calcium, magnesium and aluminum salts of fatty acids (i.e., metallic soaps), and organic sulfonates such as sodium lauryl sulfate. Synthetic cationic agents include, for example, cetyltrimethylammonium bromide, while synthetic nonionic agents are exemplified by glyceryl esters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The adjuvant MONTANIDE™ will also find use herein. Suitable animal oils include, for example, cod liver oil, halibut oil, menhaden oil, orange roughy oil and shark liver oil, all of which are available commercially. Suitable vegetable oils include, without limitation, canola oil, almond oil, cottonseed oil, corn oil, olive oil, peanut oil, safflower oil, sesame oil, soybean oil, and the like.

Alternatively, a number of aliphatic nitrogenous bases can be used as adjuvants with the vaccine formulations. For example, known immunologic adjuvants include amines, quaternary ammonium compounds, guanidines, benzamidines and thiouroniums (Gall, D. (1966) Immunology 11:369 386). Specific compounds include dimethyldioctadecylammonium bromide (DDA) (available from Kodak) and N,N-dioctadecyl-N,N-bis(2-hydroxyethyl)propanediamine (“AVRIDINE”). The use of DDA as an immunologic adjuvant has been described; See, e.g., the Kodak Laboratory Chemicals Bulletin 56(1): 1 5 (1986); Adv. Drug Deliv. Rev. 5(3):163 187 (1990); J. Controlled Release 7:123 132 (1988); Clin. Exp. Immunol. 78(2):256 262 (1989); J. Immunol. Methods 97(2): 159 164 (1987); Immunology 58(2):245 250 (1986); and Int. Arch. Allergy Appl. Immunol. 68(3):201 208 (1982). AVRIDINE is also a well-known adjuvant. See, e.g., U.S. Pat. No. 4,310,550, incorporated herein by reference in its entirety, which describes the use of N,N-higher alkyl-N′, N′-bis(2-hydroxyethyl)propane diamines in general, and AVRIDINE in particular, as vaccine adjuvants. U.S. Pat. No. 5,151,267 to Babiuk, incorporated herein by reference in its entirety, and Babiuk et al. (1986) Virology 159:57 66, also relate to the use of AVRIDINE as a vaccine adjuvant.

Once prepared, the formulations will contain a “pharmaceutically effective amount” of the active ingredient, that is, an amount capable of achieving the desired response in a subject to which the composition is administered. In the treatment and prevention of an RSV or PIV3 disease, a “pharmaceutically effective amount” would preferably be an amount which prevents, reduces or ameliorates the symptoms of the disease in question. The exact amount is readily determined by one skilled in the art using standard tests. The active ingredient will typically range from about 1% to about 95% (w/w) of the composition, or even higher or lower if appropriate. With the present formulations, 1 μg to 2 mg, such as 10 μg to 1 mg, e.g., 25 μg to 0.5 mg, 50 μg to 500 μg, or any values between these ranges of active ingredient per mL of injected solution should be adequate to treat or prevent infection when a dose of 0.5 to 2 mL per subject is administered. The quantity to be administered depends on the subject to be treated, the capacity of the subject's immune system to synthesize antibodies, and the degree of protection desired. Effective dosages can be readily established by one of ordinary skill in the art through routine trials establishing dose response curves.

The composition can be administered parenterally, e.g., by intratracheal, intramuscular, sub-cutaneous, intraperitoneal, or intravenous injection. The subject is administered at least one dose of the composition. Moreover, the subject may be administered as many doses as is required to bring about the desired biological effect.

Additional formulations which are suitable for other modes of administration include sup-positories and, in some cases, aerosol, intranasal, oral formulations, and sustained release formulations. For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject antigens by the nasal mucosa.

Controlled or sustained release formulations are made by incorporating the antigen into carriers or vehicles such as liposomes, nonresorbable impermeable polymers such as ethylenevinyl acetate copolymers and HYTREL copolymers, swellable polymers such as hydrogels, resorbable polymers such as collagen and certain polyacids or polyesters such as those used to make resorbable sutures, polyphosphazenes, alginate, microparticles, gelatin nanospheres, chitosan nanoparticles, and the like. The antigens described herein can also be delivered using implanted mini-pumps, well known in the art.

The vaccine can also be administered to any age group, including adults, children, newborns, or elderly, or pregnant women as an approach to maternal immunization.

Prime-boost methods can be employed where one or more compositions are delivered in a “priming” step and, subsequently, one or more compositions are delivered in a “boosting” step. In certain embodiments, priming and boosting with one or more compositions described herein is followed by additional boosting. The compositions delivered can include the same antigens, or different antigens, given in any order and via any administration route.

### E. Tests to Determine the Efficacy of an Immune Response

One way of assessing the efficacy of prophylactic treatment involves monitoring antibody responses against the RSV and/or PIV3 antigens in the compositions of the invention after administration of the composition, either by testing the subject's sera by ELISA, by virus neutralization assays or by screening the subject's sera by immunoblot. A positive reaction indicates that the subject has previously mounted an immune response to the RSV and PIV3 antigens, that is, the RSV- PIV3 protein is an immunogen. This method may also be used to identify epitopes. Cell-mediated immune responses, i.e. antigen-induced CD4 and/or CD8 T cells, can be measured by ELISPOT assays or flow cytometry.

Another way of checking the efficacy of prophylactic treatment involves monitoring infection with either RSV or PIV3 after administration of the compositions of the invention. The immunogenic compositions may or may not be derived from the same strains as the viral challenge strains. Preferably the immunogenic compositions are derivable from the same viral strains as the challenge strains. One way of checking the efficacy of prophylactic treatment involves monitoring immune responses both systemically (such as monitoring the level of IgG1 and IgG2a production) and mucosally (such as monitoring the level of IgA production) against the antigens in the compositions of the invention after administration of the composition. Typically, serum-specific antibody responses are determined post-immunization but pre-challenge, whereas mucosal specific antibody responses are determined post-immunization and post-challenge.

The immunogenic compositions of the present invention may be evaluated in in vitro and in vivo animal models prior to human administration.

The immune response may be one or both of a TH1-type immune response and a TH2-type response. The immune response may be an improved or an enhanced or an altered immune response. The immune response may be one or both of a systemic and a mucosal immune response. An enhanced systemic and/or mucosal immunity is reflected in an enhanced TH1-type and/or TH2-type immune response. Preferably, the enhanced immune response includes an increase in the production of IgG1 and/or IgG2a and/or IgA.

Activated TH2 cells enhance antibody production and are therefore of value in responding to extracellular infections. Activated TH2 cells may secrete one or more of IL-4, IL-5, IL-6, and IL-10. A TH2-type immune response may result in the production of IgG1, IgE, IgA and memory B cells for future protection. Generally, the enhanced TH2-type immune response will include an increase in IgG1 production.

A TH1-type immune response may include one or more of an increase in CTLs, an increase in one or more of the cytokines associated with a TH1-type immune response (such as IL-2, IFNγ, and TNF-α), an increase in activated macrophages, an increase in NK activity, or an increase in the production of IgG2a. Generally, the enhanced TH1-type immune response will include an increase in IgG2a production.

The immunogenic compositions of the present invention will preferably induce long lasting immunity that can quickly respond upon exposure to one or more infectious agents.

In case of a therapeutic application, a composition of the invention will be administered after infection with either RSV or PIV3, followed by monitoring of the progression of infection.

### F. Cyclopolyphosphazenes

Below are examples of cyclic polyphosphazenes that may be used in the compositions disclosed herein. Supporting data for the methods of preparation, testing and uses of cyclopolyphosphazenes of formula I can be found in U.S. provisional application no. 63/178,214, filed on Apr. 22, 2021, titled CYCLOPOLYPHOSPHAZENES, RELATED METHODS OF PREPARATION AND METHODS OF USE, the entirety of which is incorporated herein by reference.

Disclosed herein are compounds of formula I:

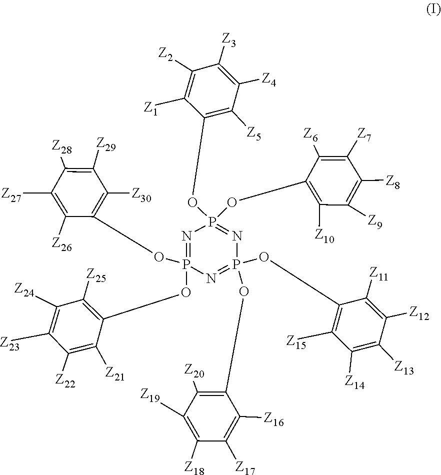

and tautomers, stereoisomers, polymorphs, hydrates, solvates, or pharmaceutically acceptable salts thereof.

In embodiments disclosed herein, each of Z₁, Z₂, Z₃, Z₄, Z₅, Z₆, Z₇, Z₈, Z₉, Z₁₀, Z₁₁, Z₁₂, Z₁₃, Z₁₄, Z₁₅, Z₁₆, Z₁₇, Z₁₈, Z₁₉, Z₂₀, Z₂₁, Z₂₂, Z₂₃, Z₂₄, Z₂₅, Z₂₆, Z₂₇, Z₂₈, Z₂₉, and Z₃₀, hereinto referred to as Z₁₋₃₀, may be independently selected from: H or formula II:

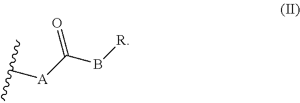

In some embodiments, at least one of Z₁₋₃₀ is substituted with formula II. Each formula II substitution of Z₁₋₃₀ may be identical or non-identical.

It will be understood by a person of skill in the art that “each of Z₁₋₃₀ may be identical or non-identical” may refer to embodiments in which each of the Z₁₋₃₀ may be represented by non-identical groups of formula II. Each A, B and/or R groups may be the same between one or more Z₁₋₃₀ substitutions or unique from one another. For example, Z₁ may be substituted with formula II wherein the A group is an oxygen atom and Z₁₃ may be substituted with formula II wherein the A group is a nitrogen atom.

In embodiments disclosed herein, one or more A-groups. if present, is selected from C₁-C₇ alkyl, C₂-C₇ alkenyl, C₂-C₇ alkynyl, O, S, and N. C₁-C₇ alkyl, C₂-C₇ alkenyl, and/or C₂-C₇ alkynyl may be straight or branched and optionally substituted by one or more substituents selected from: 1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH-Boc).

It will be understood by a person of skill in the art that “if present” refers to groups that are optional within the entire structure. In embodiments in which the group is present, the connectivity of the surrounding groups are as depicted. In embodiments where the group, for example “A” in formula II, is absent, the flanking groups would be directly connected to one another. In such cases, the carbonyl of formula II would be directly connected to the appropriate Z-group position, such as Z₁.

In embodiments disclosed herein, one or more B-groups may be selected from C₁-C₇ alkyl, C₂-C₇ alkenyl, C₂-C₇ alkynyl, H, O, S, and N, wherein C₁-C₇ alkyl, C₂-C₇ alkenyl, and/or C₂-C₇ alkynyl are straight or branched and optionally substituted by one or more substituents selected from: 1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH-Boc).

In embodiments disclosed herein, one or more R-groups (sometimes referred to herein as “ligands”), if present, is selected from H, C₁-C₄₅ alkyl, C₂-C₄₅ alkenyl, and C₂-C₄₅ alkynyl, wherein C₁-C₄₅ alkyl, C₂-C₄₅ alkenyl, and/or C₂-C₄₅ alkynyl are straight or branched and optionally substituted. The one or more R-groups may comprise one or more substituents selected from: 1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH-Boc). In some embodiments described herein, R-groups may be selected from Table 1.

TABLE 1
Examples of possible R-groups (ligands).

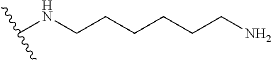 [SMILES: *NCCCCCCN]

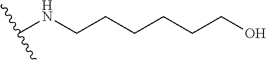 [SMILES: *NCCCCCCO]

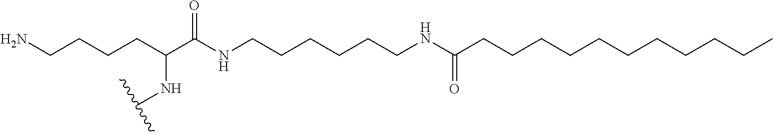 [SMILES: *NC(CCCCN)C(=O)NCCCCCCNC(=O)CCCCCCCCCCC]

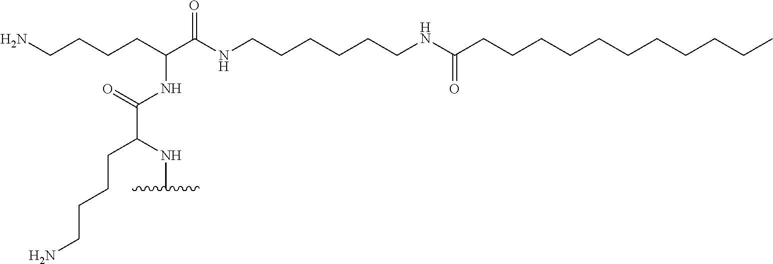 [SMILES: *NC(CCCCN)C(=O)NC(CCCCN)C(=O)NCCCCCCNC(=O)CCCCCCCCCCC]

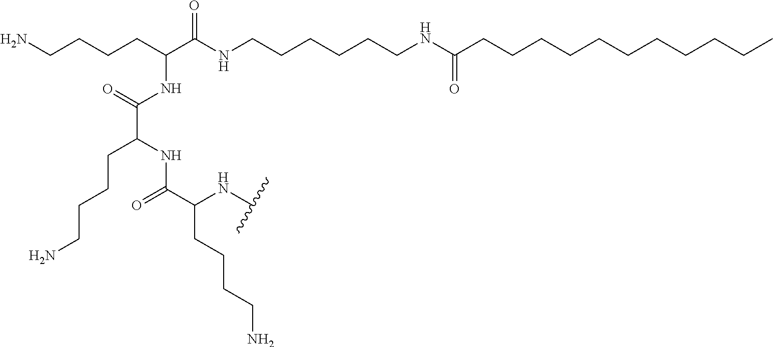 [SMILES: *NC(CCCCN)C(=O)NC(CCCCN)C(=O)NC(CCCCN)C(=O)NCCCCCCNC(=O)CCCCCCCCCCC]

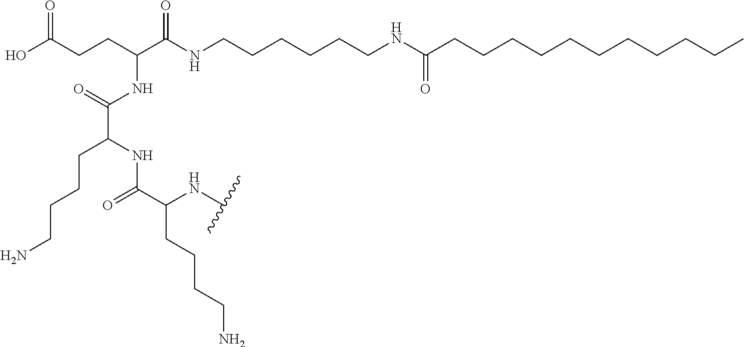 [SMILES: *NC(CCCCN)C(=O)NC(CCCCN)C(=O)NC(CCC(=O)O)C(=O)NCCCCCCNC(=O)CCCCCCCCCCC]

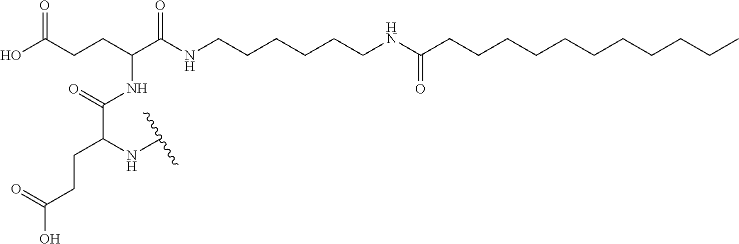 [SMILES: *NC(CCC(=O)O)C(=O)NC(CCC(=O)O)C(=O)NCCCCCCNC(=O)CCCCCCCCCCC]

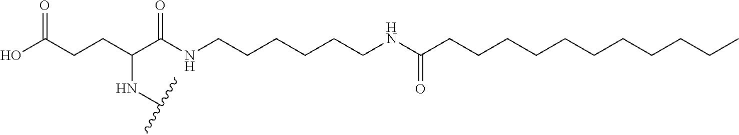 [SMILES: *NC(CCC(=O)O)C(=O)NCCCCCCNC(=O)CCCCCCCCCCC]

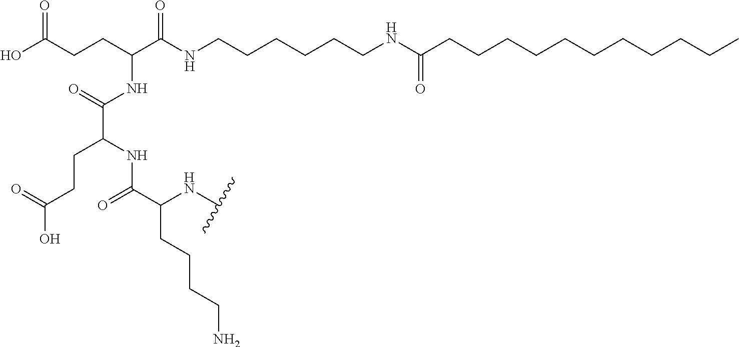 [SMILES: *NC(CCCCN)C(=O)NC(CCC(=O)O)C(=O)NC(CCC(=O)O)C(=O)NCCCCCCNC(=O)CCCCCCCCCCC]

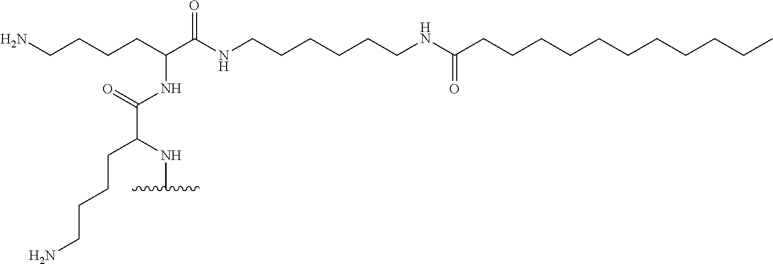 [SMILES: *NC(CCCCN)C(=O)NC(CCCCN)C(=O)NCCCCCCNC(=O)CCCCCCCCCCC]

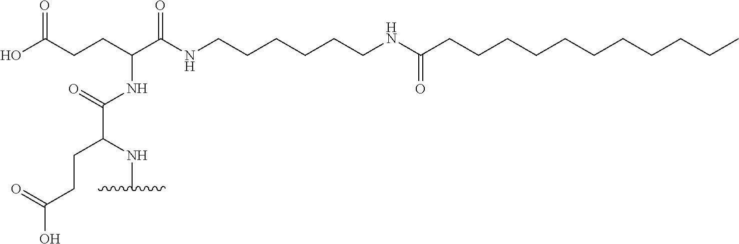 [SMILES: *NC(CCC(=O)O)C(=O)NC(CCC(=O)O)C(=O)NCCCCCCNC(=O)CCCCCCCCCCC]

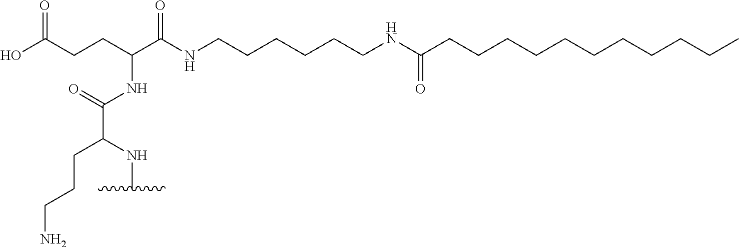 [SMILES: *NC(CCCN)C(=O)NC(CCC(=O)O)C(=O)NCCCCCCNC(=O)CCCCCCCCCCC]

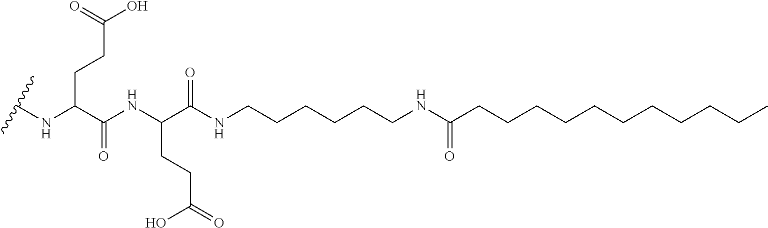 [SMILES: *NC(CCC(=O)O)C(=O)NC(CCC(=O)O)C(=O)NCCCCCCNC(=O)CCCCCCCCCCC]

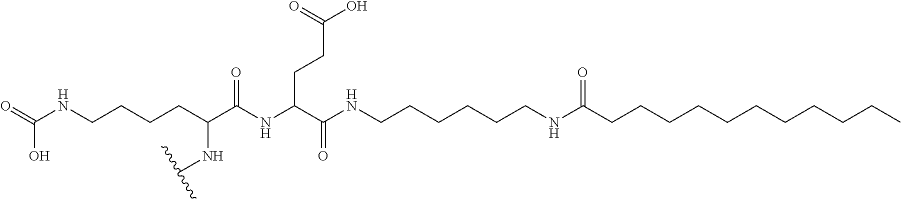 [SMILES: *NC(CCCCNC(=O)O)C(=O)NC(CCC(=O)O)C(=O)NCCCCCCNC(=O)CCCCCCCCCCC]

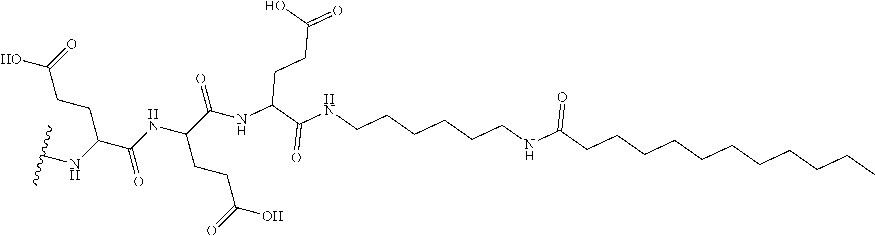 [SMILES: *NC(CCC(=O)O)C(=O)NC(CCC(=O)O)C(=O)NC(CCC(=O)O)C(=O)NCCCCCCNC(=O)CCCCCCCCCCC]

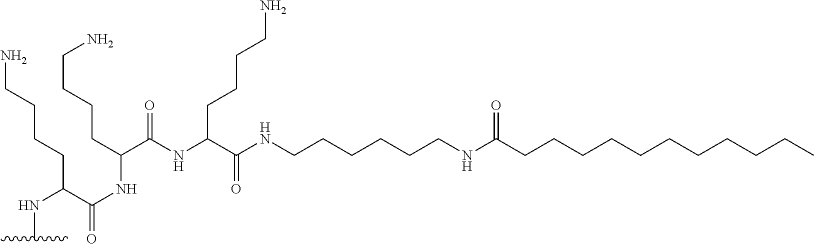 [SMILES: *NC(CCCCN)C(=O)NC(CCCCN)C(=O)NC(CCCCN)C(=O)NCCCCCCNC(=O)CCCCCCCCCCC]

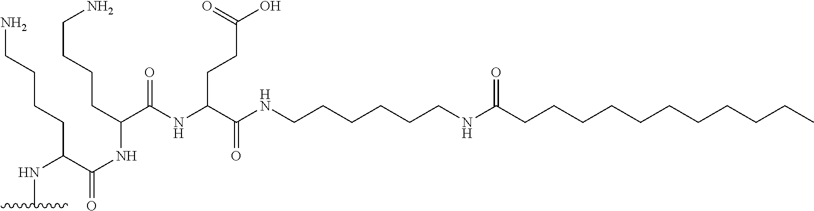 [SMILES: *NC(CCCCN)C(=O)NC(CCCCN)C(=O)NC(CCC(=O)O)C(=O)NCCCCCCNC(=O)CCCCCCCCCCC]

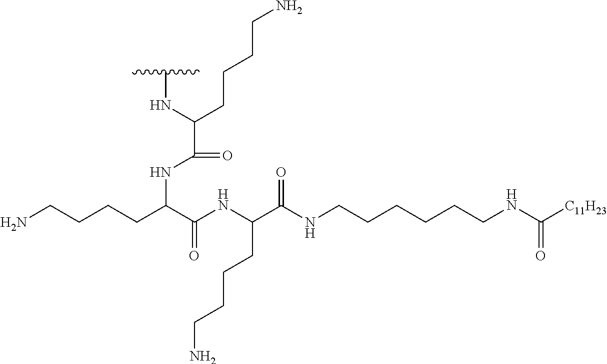

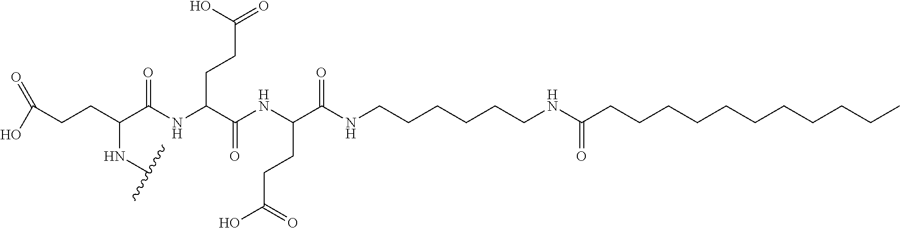 [SMILES: *NC(CCC(=O)O)C(=O)NC(CCC(=O)O)C(=O)NC(CCC(=O)O)C(=O)NCCCCCCNC(=O)CCCCCCCCCCC]

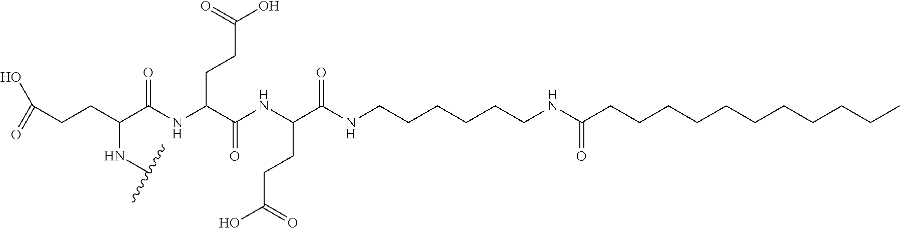 [SMILES: *NC(CCC(=O)O)C(=O)NC(CCC(=O)O)C(=O)NC(CCC(=O)O)C(=O)NCCCCCCNC(=O)CCCCCCCCCCC]

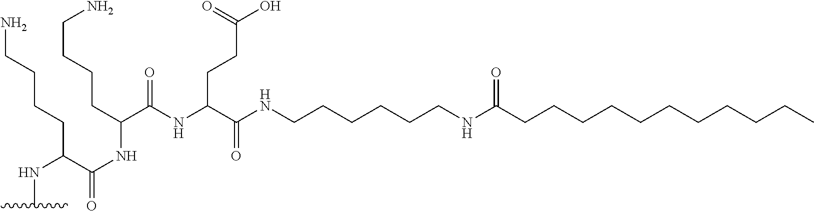 [SMILES: *NC(CCCCN)C(=O)NC(CCCCN)C(=O)NC(CCC(=O)O)C(=O)NCCCCCCNC(=O)CCCCCCCCCCC]

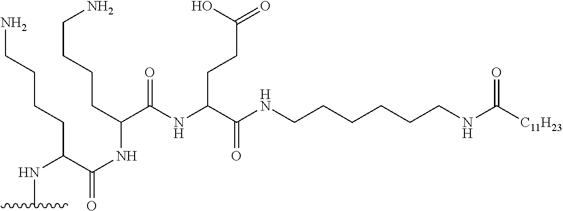

In embodiments disclosed herein, cyclopolyphosphazenes may be in various tautomeric forms, stereoisomers, polymorphs, hydrates, solvates, or pharmaceutically acceptable salts thereof.

In embodiments disclosed herein, oligomeric structures may comprise two or more cyclopolyphosphazenes compounds as defined herein. It will be understood by a person of skill in the art that “oligomeric” may refer to repeating units of either identical or non-identical cyclopolyphosphazenes that are covalently linked. Embodiments where two identical cyclopolyphosphazenes are linked may be referred to as a homodimer. Embodiments where three non-identical cyclopolyphosphazenes are linked may be referred to as a heterotrimer. Oligomeric structures may comprise two or more units, including 3, 4, 5, 6, 7, 8, 9, 10, 11, 12, 13, 14, 15 or greater units.

As disclosed herein, the two or more compounds in an oligomeric structure may be linked via a suitable connection. In some cases, the suitable connection is an amide or ester bond. The amide or ester bond may be formed via an activated ester, such as an N-hydroxy succinimide ester, and a nucleophilic group, such as a free hydroxyl or amine group. In some cases, each cyclopolyphosphazene unit in the oligomeric structure comprises an activated ester and a nucleophilic group for self-assembly into large oligomeric structures.

In some embodiments described herein, the cyclopolyphosphazene is a compound having formula 37:

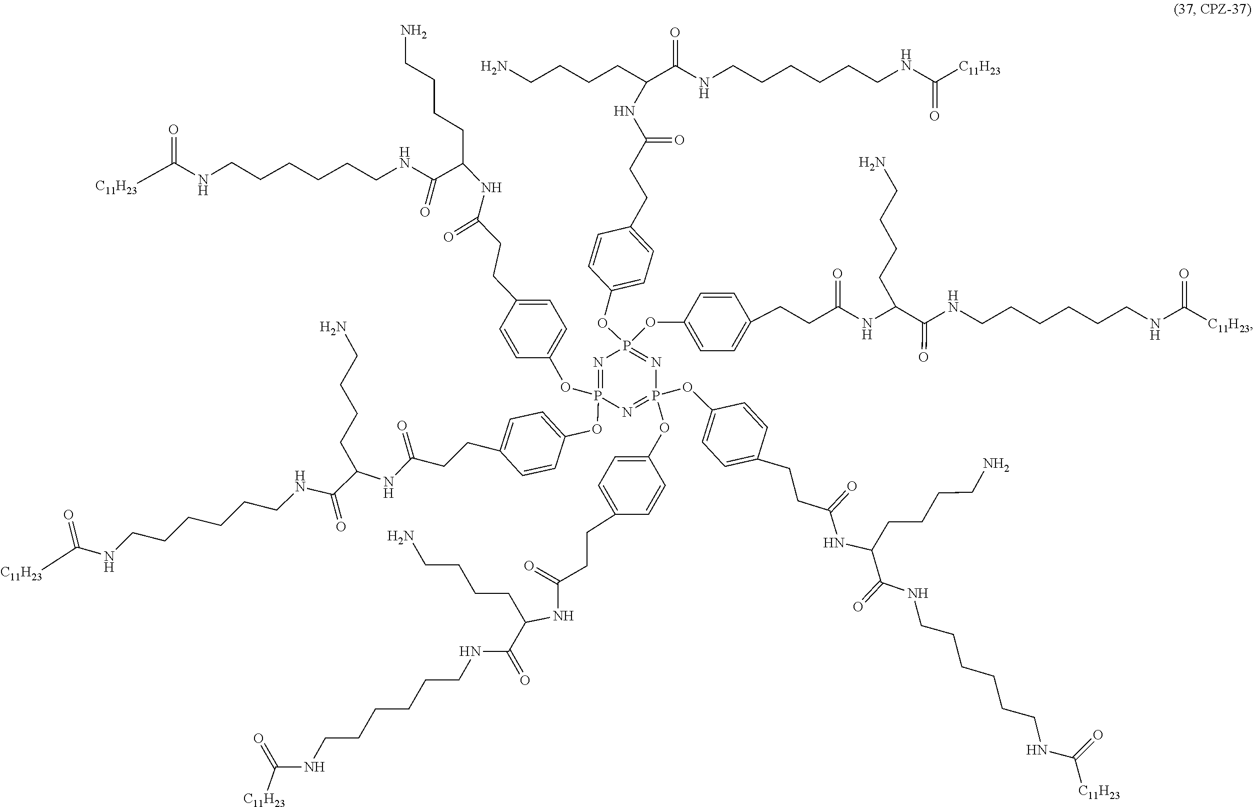

a tautomer, stereoisomer, polymorph, hydrate, solvate, or pharmaceutically acceptable salt thereof. Compound 37 may be referred to by the designation CPZ37 herein.

In some embodiments described herein, the cyclopolyphosphazene is a compound having formula 6:

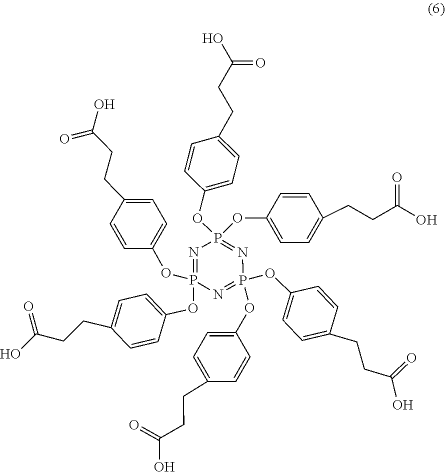 [SMILES: O=C(O)CCc1ccc(OP2(Oc3ccc(CCC(=O)O)cc3)=NP(Oc3ccc(CCC(=O)O)cc3)(Oc3ccc(CCC(=O)O)cc3)=NP(Oc3ccc(CCC(=O)O)cc3)(Oc3ccc(CCC(=O)O)cc3)=N2)cc1]

a tautomer, stereoisomer, polymorph, hydrate, solvate, or pharmaceutically acceptable salt thereof.

In some embodiments described herein, the cyclopolyphosphazene is a compound having formula 11:

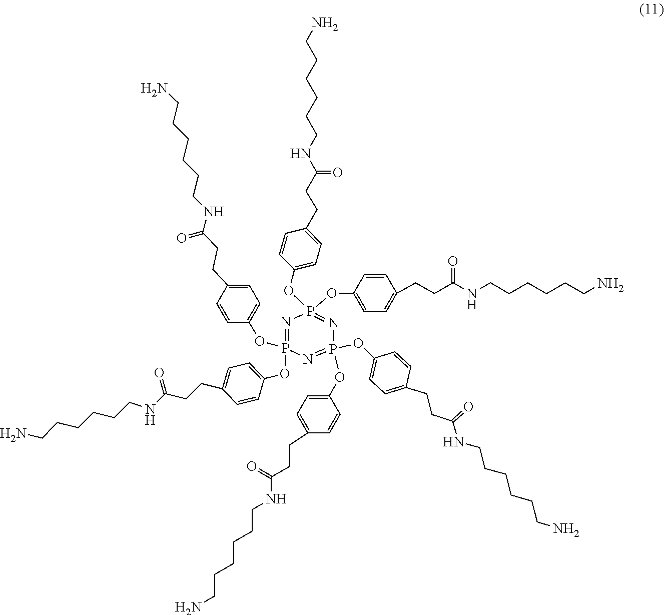 [SMILES: NCCCCCCNC(=O)CCc1ccc(OP2(Oc3ccc(CCC(=O)NCCCCCCN)cc3)=NP(Oc3ccc(CCC(=O)NCCCCCCN)cc3)(Oc3ccc(CCC(=O)NCCCCCCN)cc3)=NP(Oc3ccc(CCC(=O)NCCCCCCN)cc3)(Oc3ccc(CCC(=O)NCCCCCCN)cc3)=N2)cc1]

a tautomer, stereoisomer, polymorph, hydrate, solvate, or pharmaceutically acceptable salt thereof.

In some embodiments described herein, the cyclopolyphosphazene is a compound having formula 9:

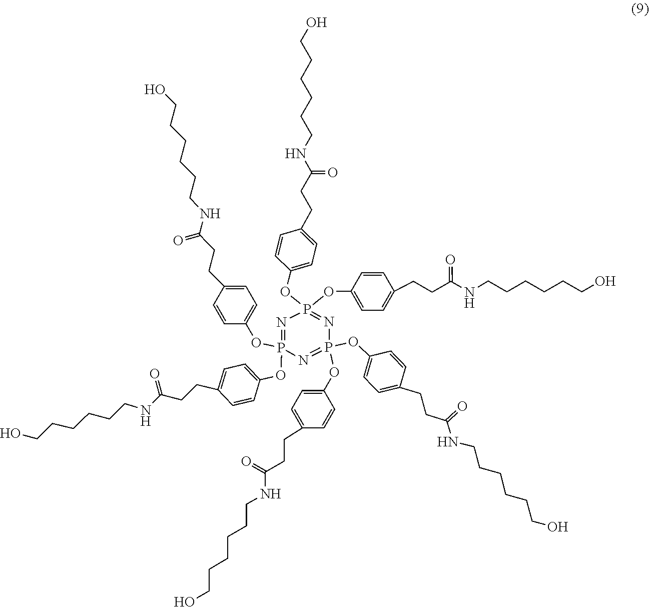 [SMILES: O=C(CCc1ccc(OP2(Oc3ccc(CCC(=O)NCCCCCCO)cc3)=NP(Oc3ccc(CCC(=O)NCCCCCCO)cc3)(Oc3ccc(CCC(=O)NCCCCCCO)cc3)=NP(Oc3ccc(CCC(=O)NCCCCCCO)cc3)(Oc3ccc(CCC(=O)NCCCCCCO)cc3)=N2)cc1)NCCCCCCO]

a tautomer, stereoisomer, polymorph, hydrate, solvate, or pharmaceutically acceptable salt thereof.

In some embodiments described herein, the cyclopolyphosphazene is a compound having formula 39:

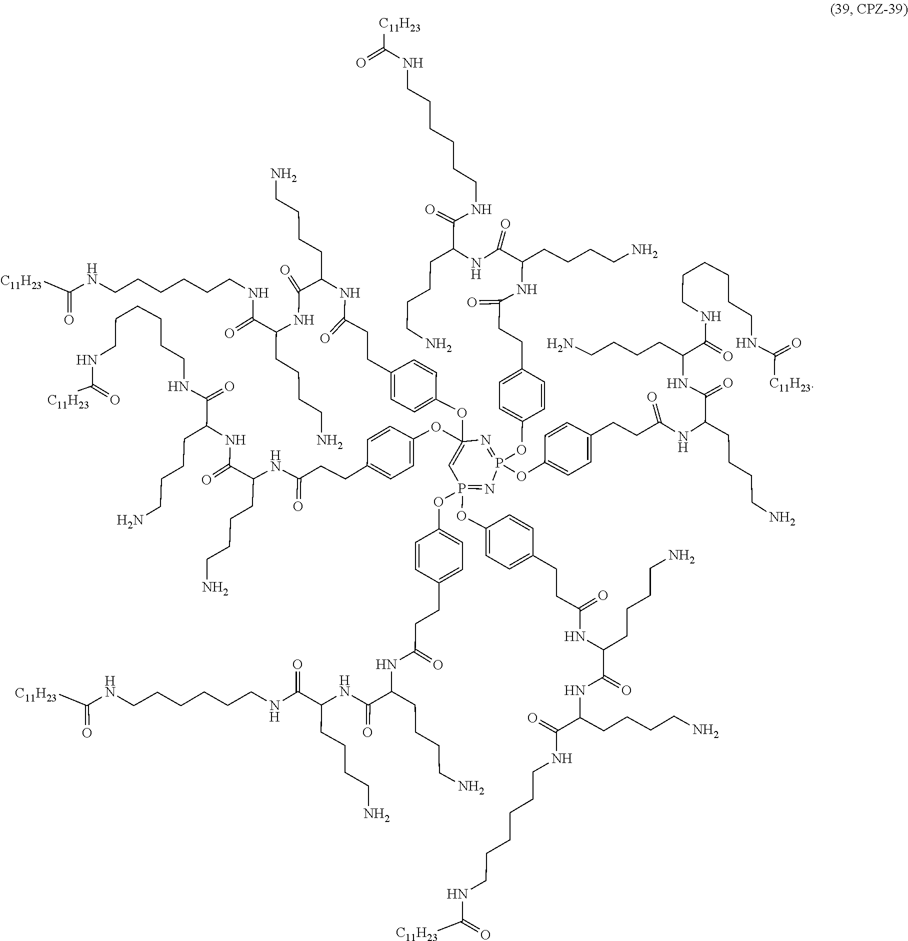

a tautomer, stereoisomer, polymorph, hydrate, solvate, or pharmaceutically acceptable salt thereof. Compound 39 may be referred to by the designation CPZ39 herein.

It will be understood to a person of skill in the art that any of the functional groups or substitutions described herein may be protected by a suitable protecting group (PG). For example, amines may be protected by a t-butyl carbamate (Boc) group. Other examples of protecting groups include: 9-Fluorenylmethyl carbamate (Fmoc), benzyl carbamate (Cbz), acyl, trifluoroacyl, phthalimide, benzyl (Bn), p-toluenesulfonamide, dithiane, acetal (cyclic or acyclic), hydrazone, alkyl or aryl esters, allyl, methoxymethyl ether (MOM ether), alkyl silyl groups (such as TBDMS and others), tetrahydropyranyl (THP) and others known in the art. Protecting groups may be removed via suitable deprotection conditions known in the art.

It will be understood by a person of skill in the art that C₁-C₇ alkyl or C₁-C₄₅ alkyl, in these embodiments as referred to herein may refer to an alkyl group between one and seven carbons. This may be understood to include straight or branched alkyl groups including for example: methyl, ethyl, isopropyl, n-propyl, tert-butyl, n-butyl, sec-butyl and others. Alkyl chains greater than seven carbons are also contemplated, for example alkyl chains of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 and greater carbons.

It will be understood by a person of skill in the art that C₂-C₇ alkenyl or C₂-C₄₅ alkenyl in these embodiments as referred to herein may refer to an alkyl group between one and seven carbons comprising at least one double bond. This may be understood to include straight or branched alkenyl groups comprising at least one double bond. C₁-C₇ alkenyl may refer to chains with two or more double bonds in conjugation. Alkenyl chains greater than seven carbons are also contemplated, for example alkenyl chains of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 and greater carbons.

It will be understood by a person of skill in the art that C₂-C₇ alkynyl or C₂-C₄₅ alkynyl in these embodiments as referred to herein may refer to an alkyl group between one and seven carbons comprising at least one triple bond. This may be understood to include straight or branched alkynyl groups comprising at least one triple bond. C₁-C₇ alkynyl may refer to chains with two or more triple bonds in conjugation. Alkynyl chains greater than seven carbons are also contemplated, for example alkynyl chains of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 and greater carbons.

The C₁-C₇ alkyl, C₁-C₄₅ alkyl, C₂-C₇ alkenyl, C₂-C₄₅ alkenyl, C₂-C₇ alkynyl, and/or C₂-C₄₅ alkynyl may be substituted by one or more suitable substituents. For example, each carbon of the one or more C₁-C₇ alkyl, C₁-C₃₀ alkyl, C₂-C₇ alkenyl, C₂-C₃₀ alkenyl, C₂-C₇ alkynyl, and/or C₂-C₃₀ alkynyl chain may be substituted with one or more of: hydroxyl, aldehyde, 1° amino, 2° amino, 3° amino, tert-butyloxycarbonyl (—NH-Boc), cyano, amide, aryl, alkoxy, acetal, ketone, ester, acyl, ether, thioether, thioester, thiol, disulfide, peroxide, imine, imide, oxime, acyl halide, nitro, nitrile, epoxide, sulfonic acid, sulphonamidyl, carbamimidoyl, azide and others.

It will be understood by a person of skill in the art that sulfonamidyl may be understood as a sulfonamide group connected to the parent group, with a nitrogen optionally substituted by 0-2 suitable substituents, such as an alkyl, aryl and others.

It will be understood by a person of skill in the art that a primary (1°), secondary (2°), tertiary (3°), and quaternary (4°) amino groups may refer to an amine with 0, 1, 2 and 3 additional substituents (apart from the parent group), respectfully. Substituents may be alkyl, alkenyl, alkynyl, aryl and others without departing from what is contemplated by the invention. It will be understood by a person of skill in the art that an amide as listed in the claims may be part of the backbone of the alkyl chain or a substituent thereof. Amides may be primary (1°), secondary (2°), or tertiary (3°).

It will be understood by a person of skill in the art that an aryl group may refer to any suitable aromatic ring or rings, such as aromatic hydrocarbons and heterocyclic rings. Examples include benzene (phenyl), benzyl, naphthalene (naphthyl), anthracene (anthracenyl), pyrene (pyrenyl), indene, biphenyl, phenanthrene, pyridine, imidazole, furan, picolinyl, azole, morphorline (morpholinyl), benzathiazole, thiazole and others.

It will be understood by a person of skill in the art that an alkoxy group refers to an ether bond comprising an alkyl group, such as a C₁-C₄₅ alkyl, C₁-C₄₅ alkenyl, C₁-C₄₅ alkylyl chain, connected to the parent group. Ether bonds may connect other non-alkyl groups, such as an aryl group, to a parent group.

It will be understood by a person of skill in the art that an acetal may refer to two geminal alkoxy groups. A hemiacetal will be understood as an alkoxy group connected at the same carbon as a hydroxyl group.

It will be understood by a person of skill in the art that an acyl or alkanoyl group may refer to an alkyl or aryl group connected to a parent group via a ketone. Acyl halide may refer to acyl group that comprises a carbonyl bonded to a halide, such as acyl chloride or acyl bromide.

It will be understood by a person of skill in the art that a halo group or halide may refer to any suitable halogen, for example fluorine (F), bromine (Br), iodine (I) and others.

Typical amounts of polyphosphazene present in the adjuvant compositions will represent from about 0.01 to about 2500 μg/kg, typically from about 0.05 to about 500 μg/kg, such as from 0.5 to 100 μg/kg, or 1 to 50 μg/kg, or any amount within these values. One of skill in the art can determine the amount of polyphosphazene, as well as the ratio of polyphosphazene to the other components in the adjuvant composition.

### G. Kits

The invention also provides kits comprising one or more containers of compositions of the invention. Compositions can be in liquid form or can be lyophilized, as can individual antigens. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may further include a third component comprising an adjuvant.

The kit can also comprise a package insert containing written instructions for methods of inducing immunity or for treating infections. The package insert can be an unapproved draft package insert or can be a package insert approved by the Food and Drug Administration (FDA) or other regulatory body.

The invention also provides a delivery device pre-filled with the immunogenic compositions of the invention.

Similarly, antibodies can be provided in kits, with suitable instructions and other necessary reagents. The kit can also contain, depending on if the antibodies are to be used in immunoassays, suitable labels and other packaged reagents and materials (i.e. wash buffers and the like). Standard immunoassays can be conducted using these kits.

### 3. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### A. Materials and Methods

### Expression and Purification of Recombinant Proteins

An open reading frame (ORF) was designed to express the N-terminal ectodomain, with signal peptide, of RSV F (Gen Bank Accession Number P03420 sequence; amino acids 1-513, including the following amino acid substitutions: L4P, T16A, G25S, I79M, P102A, T103A, A122T, V152I, S213R, A241V, I379V, M447V) fused with the ectodomain of human PIV3 (strain JS; nucleotide accession number Z11575; amino acids 87-572 of the third protein identified in 5′3′ frame 2 translation). The tF and HN domains were linked together by a BamHI site creating a GS linker.

In addition, DNA encoding the pep27 (amino acids 110-136) of the tF domain was deleted and furin recognition sites were mutated (substitutions are given below) so as to express a single chain variant of tF. The C terminal domain was made to contain a GSGSG(H)₁₂ histidine-tag to facilitate purification.

Table 2 lists the constructs that were prepared and their characteristics. Table 3 lists the sequences of the constructs.

TABLE 2

| | | | | Position | | |
|---|---|---|---|---|---|---|
| | | | | 510-513 of | | |
| | | | Position | P03420 & | | |
| | | | 137-142 in | position | | |
| | Position | | WT | 87-89 of | | |
| | 103-109 | Position 110-136 | RSV F | Z11575's | | C- |
| | (in GenBank | in WT RSV F | (GenBank | translated | Position | terminal |
| Construct | P03420) | GenBank P03420) | P03420) | sequence | 979 | addition |
| WT | ANNRAR | pep27 | FLGF | DELL | N/A | N/A |
| RSV F | R | (ELPRFMNYTLNNTKKTN | LL | | | |
| sequence | | VTLSKKRKRR) | | | | |
| GenBank | | | | | | |
| Accession | | | | | | |
| No. | | | | | | |
| P03420 | | | | | | |
| (including | | | | | | |
| the | | | | | | |
| following | | | | | | |
| amino acid | | | | | | |
| sub- | | | | | | |
| stitutions: | | | | | | |
| L4P, | | | | | | |
| T16A, | | | | | | |
| G25S, | | | | | | |
| I79M, | | | | | | |
| P102A, | | | | | | |
| T103A, | | | | | | |
| A122T, | | | | | | |
| V152I, | | | | | | |
| S213R, | | | | | | |
| A241V, | | | | | | |
| I379V, | | | | | | |
| M447V) | | | | | | |
| tFrSc6- | ANNQAR | GSGSGR | SLGF | DELLGS | PKS | GSGSG |
| HN | | | LL | TND | CS | (H)₁₂ |
| tFrSc8- | ANNRAR | GSGSGR | TLGF | DELLGS | PKS | GSGSG |
| HN | | | LL | TND | CS | (H)₁₂ |
| tFrSc10- | ANNNAR | GSGSGR | TLGF | DELLGS | PKS | GSGSG |
| HN | | | LL | TND | CS | (H)₁₂ |

TABLE 3

| | Sequence | |
|---|---|---|
| Construct | ID No. | Sequence |
| tFrSc6- | SEQ ID | MELPILKANAITTILAAVTFCFASSQNITEEFYQSTCSAVSKGYL |
| HN | No. 1 | SALRTGWYTSVITIELSNIKENKCNGTDAKVKLMKQELDKYK |
| | | NAVTELQLLMQSTPAANNQARGSGSGRSLGFLLGVGSAIASGI |
| | | AVSKVLHLEGEVNKIKSALLSTNKAVVSLSNGVSVLTSKVLDL |
| | | KNYIDKQLLPIVNKQSCRISNIETVIEFQQKNNRLLEITREFSVN |
| | | VGVTTPVSTYMLTNSELLSLINDMPITNDQKKLMSNNVQIVRQ |
| | | QSYSIMSIIKEEVLAYVVQLPLYGVIDTPCWKLHTSPLCTTNTK |
| | | EGSNICLTRTDRGWYCDNAGSVSFFPQAETCKVQSNRVFCDT |
| | | MNSLTLPSEVNLCNVDIFNPKYDCKIMTSKTDVSSSVITSLGAI |
| | | VSCYGKTKCTASNKNRGIIKTFSNGCDYVSNKGVDTVSVGNT |
| | | LYYVNKQEGKSLYVKGEPIINFYDPLVFPSDEFDASISQVNEKI |
| | | NQSLAFIRKSDELLGSTNDLIQSGVNTRLLTIQSHVQNYIPISLT |
| | | QQMSDLRKFISEITIRNDNQEVLPQRITHDVGIKPLNPDDFWRC |
| | | TSGLPSLMKTPKIRLMPGPGLLAMPTTVDGCVRTPSLVINDLIY |
| | | AYTSNLITRGCQDIGKSYQVLQIGIITVNSDLVPDLNPRISHTFNI |
| | | NDNRKSCSLALLNTDVYQLCSTPKVDERSDYASSGIEDIVLDIV |
| | | NYDGSISTTRFKNNNISFDQPYAALYPSVGPGIYYKGKIIFLGY |
| | | GGLEHPINENVICNTTGCPGKTQRDCNQASHSPWFSDRRMVNS |
| | | IIVVDKGLNSIPKLKVWTISMRQNYWGSEGRLLLLGNKIYIYTR |
| | | STSWHSKLQLGIIDITDYSDIRIKWTWHNVLSRPGNNECPWGH |
| | | SCPDGCITGVYTDAYPLNPTGSIVSSVILDSQKSRVNPVITYSTA |
| | | TERVNELAILNRTLSAGYTTTSCITHYNKGYCFHIVEINHKSLN |
| | | TFQPMLFKTEIPKSCSGSGSGHHHHHHHHHHHH |
| tFrSc6- | SEQ ID | ATGGAGCTGCCTATCCTGAAGGCCAACGCCATCACCACAAT |
| HN | No. 2 | TCTGGCCGCCGTGACCTTCTGTTTTGCCAGCAGCCAGAACAT |
| | | CACCGAGGAGTTCTACCAGAGCACCTGTAGCGCCGTGAGCA |
| | | AGGGCTATCTGAGCGCCCTGAGAACCGGCTGGTACACCAGC |
| | | GTGATCACCATCGAGCTGAGCAACATCAAGGAGAACAAGT |
| | | GCAACGGCACCGACGCCAAGGTGAAGCTGATGAAGCAGGA |
| | | GCTGGACAAGTACAAGAACGCCGTGACCGAACTGCAGCTG |
| | | CTGATGCAGTCTACCCCTGCCGCCAACAACCAGGCGAGAGG |
| | | CAGCGGCTCTGGCAGAAGCCTGGGCTTTCTGCTGGGAGTGG |
| | | GCTCTGCCATCGCCTCTGGCATCGCCGTGTCTAAGGTGCTGC |
| | | ACCTGGAGGGAGAGGTGAACAAGATCAAGAGCGCCCTGCT |
| | | GAGCACCAATAAGGCCGTGGTGAGCCTGAGCAATGGCGTG |
| | | AGCGTGCTGACAAGCAAGGTGCTGGACCTCAAGAACTACAT |
| | | CGACAAGCAGCTGCTGCCCATCGTGAACAAGCAGAGCTGCC |
| | | GGATCAGCAACATCGAGACCGTGATCGAGTTCCAGCAGAA |
| | | GAACAACCGGCTGCTGGAGATCACCAGGGAGTTCAGCGTG |
| | | AATGTGGGCGTGACCACCCCTGTGAGCACCTACATGCTGAC |
| | | CAACAGCGAGCTGCTGAGCCTGATCAACGACATGCCCATCA |
| | | CCAACGACCAGAAGAAGCTGATGTCCAACAACGTGCAGAT |
| | | CGTGCGGCAGCAGAGCTACAGCATCATGTCCATCATCAAGG |
| | | AGGAGGTGCTGGCTTACGTGGTGCAGCTGCCTCTGTACGGC |
| | | GTGATCGACACCCCTTGCTGGAAGCTGCACACCAGCCCTCT |
| | | GTGCACCACCAATACCAAGGAGGGCAGCAACATCTGCCTGA |
| | | CCAGGACCGATAGAGGCTGGTACTGCGACAATGCCGGCAG |
| | | CGTGAGCTTCTTTCCACAGGCCGAGACCTGTAAGGTGCAGA |
| | | GCAACCGGGTGTTCTGCGACACCATGAACAGCCTGACCCTG |
| | | CCTTCTGAGGTGAACCTGTGCAACGTGGACATCTTCAACCC |
| | | CAAGTACGACTGCAAGATCATGACCAGCAAGACCGACGTG |
| | | AGCAGCAGCGTGATTACAAGCCTGGGCGCCATCGTGAGCTG |
| | | TTACGGCAAGACCAAGTGCACCGCCAGCAACAAGAACCGC |
| | | GGCATCATCAAGACCTTCAGCAACGGCTGCGACTACGTGAG |
| | | CAACAAGGGCGTGGATACAGTGAGCGTGGGCAACACCCTG |
| | | TACTACGTCAACAAGCAGGAGGGCAAGAGCCTGTACGTGA |
| | | AGGGCGAGCCCATCATCAACTTCTACGACCCCCTGGTGTTC |
| | | CCTAGCGACGAGTTCGATGCCAGCATCAGCCAGGTGAACGA |
| | | GAAGATCAACCAGAGCCTGGCCTTCATCAGGAAGAGCGAC |
| | | GAGCTGCTGGGATCCACTAACGACCTGATCCAGTCTGGCGT |
| | | GAACACAAGGCTGCTGACTATTCAGAGCCACGTCCAGAATT |
| | | ACATCCCAATTTCCCTGACACAGCAGATGTCTGACCTGAGG |
| | | AAGTTCATCTCCGAAATCACTATTCGCAATGATAACCAGGA |
| | | GGTGCTGCCCCAGCGCATCACCCATGACGTGGGCATCAAGC |
| | | CACTGAACCCCGACGATTTTTGGAGGTGCACTTCAGGACTG |
| | | CCTAGCCTGATGAAGACCCCAAAAATCCGACTGATGCCAGG |
| | | ACCTGGACTGCTGGCAATGCCAACCACAGTGGATGGATGCG |
| | | TCCGAACCCCCTCTCTGGTCATCAACGACCTGATCTACGCCT |
| | | ATACTAGTAATCTGATCACCCGCGGCTGTCAGGACATTGGG |
| | | AAGTCCTACCAGGTGCTGCAGATCGGCATCATTACAGTGAA |
| | | CAGTGATCTGGTCCCCGACCTGAATCCTCGCATCTCACACA |
| | | CTTTTAATATCAACGATAACCGAAAGTCATGCAGCCTGGCT |
| | | CTGCTGAACACAGACGTGTACCAGCTGTGCTCTACTCCTAA |
| | | AGTCGATGAACGGAGTGACTATGCAAGCTCCGGCATCGAGG |
| | | ATATTGTGCTGGACATCGTCAATTACGATGGGTCCATTTCTA |
| | | CTACCAGATTCAAGAACAATAACATCAGCTTTGACCAGCCC |
| | | TACGCCGCTCTGTATCCATCCGTGGGACCAGGAATCTACTA |
| | | CAAGGGAAAAATCATTTTCCTGGGCTATGGCGGGCTGGAAC |
| | | ACCCTATCAACGAGAATGTGATTTGCAACACAACTGGCTGT |
| | | CCAGGGAAGACCCAGAGGGATTGCAATCAGGCCAGTCATTC |
| | | ACCCTGGTTTAGTGATCGGAGAATGGTGAACTCAATCATTG |
| | | TGGTCGACAAAGGGCTGAATAGCATCCCTAAGCTGAAAGTC |
| | | TGGACCATTTCAATGCGACAGAACTACTGGGGAAGCGAAG |
| | | GCCGGCTGCTGCTGCTGGGCAATAAGATCTACATCTACACT |
| | | CGGAGCACCTCCTGGCACTCCAAACTGCAGCTGGGGATCAT |
| | | TGACATCACCGATTATTCTGACATCCGGATTAAGTGGACAT |
| | | GGCACAACGTGCTGTCAAGACCCGGGAATAACGAGTGTCCT |
| | | TGGGGACATAGCTGCCCAGATGGGTGTATCACCGGAGTGTA |
| | | CACAGACGCTTATCCTCTGAACCCAACCGGCAGTATCGTGT |
| | | CTAGTGTCATTCTGGACTCTCAGAAAAGTAGAGTGAATCCC |
| | | GTCATCACATACAGCACCGCAACAGAAAGAGTGAACGAGC |
| | | TGGCCATTCTGAATAGGACTCTGAGCGCCGGATATACCACA |
| | | ACTTCCTGCATCACCCATTACAACAAGGGCTATTGTTTCCAC |
| | | ATCGTGGAAATTAACCATAAAAGCCTGAATACCTTCCAGCC |
| | | CATGCTGTTTAAGACAGAGATTCCTAAAAGTTGTTCAGGCA |
| | | GTGGGTCAGGACATCACCATCATCATCATCATCACCATCAT |
| | | CACCATTGA |
| tFrSc8- | SEQ ID | MELPILKANAITTILAAVTFCFASSQNITEEFYQSTCSAVSKGYL |
| HN | No. 3 | SALRTGWYTSVITIELSNIKENKCNGTDAKVKLMKQELDKYK |
| | | NAVTELQLLMQSTPAANNRARGSGSGRTLGFLLGVGSAIASGI |
| | | AVSKVLHLEGEVNKIKSALLSTNKAVVSLSNGVSVLTSKVLDL |
| | | KNYIDKQLLPIVNKQSCRISNIETVIEFQQKNNRLLEITREFSVN |
| | | VGVTTPVSTYMLTNSELLSLINDMPITNDQKKLMSNNVQIVRQ |
| | | QSYSIMSIIKEEVLAYVVQLPLYGVIDTPCWKLHTSPLCTTNTK |
| | | EGSNICLTRTDRGWYCDNAGSVSFFPQAETCKVQSNRVFCDT |
| | | MNSLTLPSEVNLCNVDIFNPKYDCKIMTSKTDVSSSVITSLGAI |
| | | VSCYGKTKCTASNKNRGIIKTFSNGCDYVSNKGVDTVSVGNT |
| | | LYYVNKQEGKSLYVKGEPIINFYDPLVFPSDEFDASISQVNEKI |
| | | NQSLAFIRKSDELLGSTNDLIQSGVNTRLLTIQSHVQNYIPISLT |
| | | QQMSDLRKFISEITIRNDNQEVLPQRITHDVGIKPLNPDDFWRC |
| | | TSGLPSLMKTPKIRLMPGPGLLAMPTTVDGCVRTPSLVINDLIY |
| | | AYTSNLITRGCQDIGKSYQVLQIGIITVNSDLVPDLNPRISHTFNI |
| | | NDNRKSCSLALLNTDVYQLCSTPKVDERSDYASSGIEDIVLDIV |
| | | NYDGSISTTRFKNNNISFDQPYAALYPSVGPGIYYKGKIIFLGY |
| | | GGLEHPINENVICNTTGCPGKTQRDCNQASHSPWFSDRRMVNS |
| | | IIVVDKGLNSIPKLKVWTISMRQNYWGSEGRLLLLGNKIYIYTR |
| | | STSWHSKLQLGIIDITDYSDIRIKWTWHNVLSRPGNNECPWGH |
| | | SCPDGCITGVYTDAYPLNPTGSIVSSVILDSQKSRVNPVITYSTA |
| | | TERVNELAILNRTLSAGYTTTSCITHYNKGYCFHIVEINHKSLN |
| | | TFQPMLFKTEIPKSCSGSGSGHHHHHHHHHHHH |
| tFrSc8- | SEQ ID | ATGGAGCTGCCTATCCTGAAGGCCAACGCCATCACCACAAT |
| HN | No. 4 | TCTGGCCGCCGTGACCTTCTGTTTTGCCAGCAGCCAGAACAT |
| | | CACCGAGGAGTTCTACCAGAGCACCTGTAGCGCCGTGAGCA |
| | | AGGGCTATCTGAGCGCCCTGAGAACCGGCTGGTACACCAGC |
| | | GTGATCACCATCGAGCTGAGCAACATCAAGGAGAACAAGT |
| | | GCAACGGCACCGACGCCAAGGTGAAGCTGATGAAGCAGGA |
| | | GCTGGACAAGTACAAGAACGCCGTGACCGAACTGCAGCTG |
| | | CTGATGCAGTCTACCCCTGCCGCCAACAACAGAGCCAGAGG |
| | | CAGCGGCAGCGGCAGAACCCTGGGCTTTCTGCTGGGAGTGG |
| | | GCTCTGCCATCGCCTCTGGCATCGCCGTGTCTAAGGTGCTGC |
| | | ACCTGGAGGGAGAGGTGAACAAGATCAAGAGCGCCCTGCT |
| | | GAGCACCAATAAGGCCGTGGTGAGCCTGAGCAATGGCGTG |
| | | AGCGTGCTGACAAGCAAGGTGCTGGACCTCAAGAACTACAT |
| | | CGACAAGCAGCTGCTGCCCATCGTGAACAAGCAGAGCTGCC |
| | | GGATCAGCAACATCGAGACCGTGATCGAGTTCCAGCAGAA |
| | | GAACAACCGGCTGCTGGAGATCACCAGGGAGTTCAGCGTG |
| | | AATGTGGGCGTGACCACCCCTGTGAGCACCTACATGCTGAC |
| | | CAACAGCGAGCTGCTGAGCCTGATCAACGACATGCCCATCA |
| | | CCAACGACCAGAAGAAGCTGATGTCCAACAACGTGCAGAT |
| | | CGTGCGGCAGCAGAGCTACAGCATCATGTCCATCATCAAGG |
| | | AGGAGGTGCTGGCTTACGTGGTGCAGCTGCCTCTGTACGGC |
| | | GTGATCGACACCCCTTGCTGGAAGCTGCACACCAGCCCTCT |
| | | GTGCACCACCAATACCAAGGAGGGCAGCAACATCTGCCTGA |
| | | CCAGGACCGATAGAGGCTGGTACTGCGACAATGCCGGCAG |
| | | CGTGAGCTTCTTTCCACAGGCCGAGACCTGTAAGGTGCAGA |
| | | GCAACCGGGTGTTCTGCGACACCATGAACAGCCTGACCCTG |
| | | CCTTCTGAGGTGAACCTGTGCAACGTGGACATCTTCAACCC |
| | | CAAGTACGACTGCAAGATCATGACCAGCAAGACCGACGTG |
| | | AGCAGCAGCGTGATTACAAGCCTGGGCGCCATCGTGAGCTG |
| | | TTACGGCAAGACCAAGTGCACCGCCAGCAACAAGAACCGC |
| | | GGCATCATCAAGACCTTCAGCAACGGCTGCGACTACGTGAG |
| | | CAACAAGGGCGTGGATACAGTGAGCGTGGGCAACACCCTG |
| | | TACTACGTCAACAAGCAGGAGGGCAAGAGCCTGTACGTGA |
| | | AGGGCGAGCCCATCATCAACTTCTACGACCCCCTGGTGTTC |
| | | CCTAGCGACGAGTTCGATGCCAGCATCAGCCAGGTGAACGA |
| | | GAAGATCAACCAGAGCCTGGCCTTCATCAGGAAGAGCGAC |
| | | GAGCTGCTGGGATCCACTAACGACCTGATCCAGTCTGGCGT |
| | | GAACACAAGGCTGCTGACTATTCAGAGCCACGTCCAGAATT |
| | | ACATCCCAATTTCCCTGACACAGCAGATGTCTGACCTGAGG |
| | | AAGTTCATCTCCGAAATCACTATTCGCAATGATAACCAGGA |
| | | GGTGCTGCCCCAGCGCATCACCCATGACGTGGGCATCAAGC |
| | | CACTGAACCCCGACGATTTTTGGAGGTGCACTTCAGGACTG |
| | | CCTAGCCTGATGAAGACCCCAAAAATCCGACTGATGCCAGG |
| | | ACCTGGACTGCTGGCAATGCCAACCACAGTGGATGGATGCG |
| | | TCCGAACCCCCTCTCTGGTCATCAACGACCTGATCTACGCCT |
| | | ATACTAGTAATCTGATCACCCGCGGCTGTCAGGACATTGGG |
| | | AAGTCCTACCAGGTGCTGCAGATCGGCATCATTACAGTGAA |
| | | CAGTGATCTGGTCCCCGACCTGAATCCTCGCATCTCACACA |
| | | CTTTTAATATCAACGATAACCGAAAGTCATGCAGCCTGGCT |
| | | CTGCTGAACACAGACGTGTACCAGCTGTGCTCTACTCCTAA |
| | | AGTCGATGAACGGAGTGACTATGCAAGCTCCGGCATCGAGG |
| | | ATATTGTGCTGGACATCGTCAATTACGATGGGTCCATTTCTA |
| | | CTACCAGATTCAAGAACAATAACATCAGCTTTGACCAGCCC |
| | | TACGCCGCTCTGTATCCATCCGTGGGACCAGGAATCTACTA |
| | | CAAGGGAAAAATCATTTTCCTGGGCTATGGCGGGCTGGAAC |
| | | ACCCTATCAACGAGAATGTGATTTGCAACACAACTGGCTGT |
| | | CCAGGGAAGACCCAGAGGGATTGCAATCAGGCCAGTCATTC |
| | | ACCCTGGTTTAGTGATCGGAGAATGGTGAACTCAATCATTG |
| | | TGGTCGACAAAGGGCTGAATAGCATCCCTAAGCTGAAAGTC |
| | | TGGACCATTTCAATGCGACAGAACTACTGGGGAAGCGAAG |
| | | GCCGGCTGCTGCTGCTGGGCAATAAGATCTACATCTACACT |
| | | CGGAGCACCTCCTGGCACTCCAAACTGCAGCTGGGGATCAT |
| | | TGACATCACCGATTATTCTGACATCCGGATTAAGTGGACAT |
| | | GGCACAACGTGCTGTCAAGACCCGGGAATAACGAGTGTCCT |
| | | TGGGGACATAGCTGCCCAGATGGGTGTATCACCGGAGTGTA |
| | | CACAGACGCTTATCCTCTGAACCCAACCGGCAGTATCGTGT |
| | | CTAGTGTCATTCTGGACTCTCAGAAAAGTAGAGTGAATCCC |
| | | GTCATCACATACAGCACCGCAACAGAAAGAGTGAACGAGC |
| | | TGGCCATTCTGAATAGGACTCTGAGCGCCGGATATACCACA |
| | | ACTTCCTGCATCACCCATTACAACAAGGGCTATTGTTTCCAC |
| | | ATCGTGGAAATTAACCATAAAAGCCTGAATACCTTCCAGCC |
| | | CATGCTGTTTAAGACAGAGATTCCTAAAAGTTGTTCAGGCA |
| | | GTGGGTCAGGACATCACCATCATCATCATCATCACCATCAT |
| | | CACCATTGA |
| tFrSc10- | SEQ ID | MELPILKANAITTILAAVTFCFASSQNITEEFYQSTCSAVSKGYL |
| HN | No. 5 | SALRTGWYTSVITIELSNIKENKCNGTDAKVKLMKQELDKYK |
| | | NAVTELQLLMQSTPAANNNARGSGSGRTLGFLLGVGSAIASGI |
| | | AVSKVLHLEGEVNKIKSALLSTNKAVVSLSNGVSVLTSKVLDL |
| | | KNYIDKQLLPIVNKQSCRISNIETVIEFQQKNNRLLEITREFSVN |
| | | VGVTTPVSTYMLTNSELLSLINDMPITNDQKKLMSNNVQIVRQ |
| | | QSYSIMSIIKEEVLAYVVQLPLYGVIDTPCWKLHTSPLCTTNTK |
| | | EGSNICLTRTDRGWYCDNAGSVSFFPQAETCKVQSNRVFCDT |
| | | MNSLTLPSEVNLCNVDIFNPKYDCKIMTSKTDVSSSVITSLGAI |
| | | VSCYGKTKCTASNKNRGIIKTFSNGCDYVSNKGVDTVSVGNT |
| | | LYYVNKQEGKSLYVKGEPIINFYDPLVFPSDEFDASISQVNEKI |
| | | NQSLAFIRKSDELLGSTNDLIQSGVNTRLLTIQSHVQNYIPISLT |
| | | QQMSDLRKFISEITIRNDNQEVLPQRITHDVGIKPLNPDDFWRC |
| | | TSGLPSLMKTPKIRLMPGPGLLAMPTTVDGCVRTPSLVINDLIY |
| | | AYTSNLITRGCQDIGKSYQVLQIGIITVNSDLVPDLNPRISHTFNI |
| | | NDNRKSCSLALLNTDVYQLCSTPKVDERSDYASSGIEDIVLDIV |
| | | NYDGSISTTRFKNNNISFDQPYAALYPSVGPGIYYKGKIIFLGY |
| | | GGLEHPINENVICNTTGCPGKTQRDCNQASHSPWFSDRRMVNS |
| | | IIVVDKGLNSIPKLKVWTISMRQNYWGSEGRLLLLGNKIYIYTR |
| | | STSWHSKLQLGIIDITDYSDIRIKWTWHNVLSRPGNNECPWGH |
| | | SCPDGCITGVYTDAYPLNPTGSIVSSVILDSQKSRVNPVITYSTA |
| | | TERVNELAILNRTLSAGYTTTSCITHYNKGYCFHIVEINHKSLN |
| | | TFQPMLFKTEIPKSCSGSGSGHHHHHHHHHHHH |
| tFrSc10- | SEQ ID | ATGGAGCTGCCTATCCTGAAGGCCAACGCCATCACCACAAT |
| HN | No. 6 | TCTGGCCGCCGTGACCTTCTGTTTTGCCAGCAGCCAGAACAT |
| | | CACCGAGGAGTTCTACCAGAGCACCTGTAGCGCCGTGAGCA |
| | | AGGGCTATCTGAGCGCCCTGAGAACCGGCTGGTACACCAGC |
| | | GTGATCACCATCGAGCTGAGCAACATCAAGGAGAACAAGT |
| | | GCAACGGCACCGACGCCAAGGTGAAGCTGATGAAGCAGGA |
| | | GCTGGACAAGTACAAGAACGCCGTGACCGAACTGCAGCTG |
| | | CTGATGCAGTCTACCCCTGCCGCCAACAACAACGCCAGAGG |
| | | CAGCGGCAGCGGCAGAACCCTGGGCTTTCTGCTGGGAGTGG |
| | | GCTCTGCCATCGCCTCTGGCATCGCCGTGTCTAAGGTGCTGC |
| | | ACCTGGAGGGAGAGGTGAACAAGATCAAGAGCGCCCTGCT |
| | | GAGCACCAATAAGGCCGTGGTGAGCCTGAGCAATGGCGTG |
| | | AGCGTGCTGACAAGCAAGGTGCTGGACCTCAAGAACTACAT |
| | | CGACAAGCAGCTGCTGCCCATCGTGAACAAGCAGAGCTGCC |
| | | GGATCAGCAACATCGAGACCGTGATCGAGTTCCAGCAGAA |
| | | GAACAACCGGCTGCTGGAGATCACCAGGGAGTTCAGCGTG |
| | | AATGTGGGCGTGACCACCCCTGTGAGCACCTACATGCTGAC |
| | | CAACAGCGAGCTGCTGAGCCTGATCAACGACATGCCCATCA |
| | | CCAACGACCAGAAGAAGCTGATGTCCAACAACGTGCAGAT |
| | | CGTGCGGCAGCAGAGCTACAGCATCATGTCCATCATCAAGG |
| | | AGGAGGTGCTGGCTTACGTGGTGCAGCTGCCTCTGTACGGC |
| | | GTGATCGACACCCCTTGCTGGAAGCTGCACACCAGCCCTCT |
| | | GTGCACCACCAATACCAAGGAGGGCAGCAACATCTGCCTGA |
| | | CCAGGACCGATAGAGGCTGGTACTGCGACAATGCCGGCAG |
| | | CGTGAGCTTCTTTCCACAGGCCGAGACCTGTAAGGTGCAGA |
| | | GCAACCGGGTGTTCTGCGACACCATGAACAGCCTGACCCTG |
| | | CCTTCTGAGGTGAACCTGTGCAACGTGGACATCTTCAACCC |
| | | CAAGTACGACTGCAAGATCATGACCAGCAAGACCGACGTG |
| | | AGCAGCAGCGTGATTACAAGCCTGGGCGCCATCGTGAGCTG |
| | | TTACGGCAAGACCAAGTGCACCGCCAGCAACAAGAACCGC |
| | | GGCATCATCAAGACCTTCAGCAACGGCTGCGACTACGTGAG |
| | | CAACAAGGGCGTGGATACAGTGAGCGTGGGCAACACCCTG |
| | | TACTACGTCAACAAGCAGGAGGGCAAGAGCCTGTACGTGA |
| | | AGGGCGAGCCCATCATCAACTTCTACGACCCCCTGGTGTTC |
| | | CCTAGCGACGAGTTCGATGCCAGCATCAGCCAGGTGAACGA |
| | | GAAGATCAACCAGAGCCTGGCCTTCATCAGGAAGAGCGAC |
| | | GAGCTGCTGGGATCCACTAACGACCTGATCCAGTCTGGCGT |
| | | GAACACAAGGCTGCTGACTATTCAGAGCCACGTCCAGAATT |
| | | ACATCCCAATTTCCCTGACACAGCAGATGTCTGACCTGAGG |
| | | AAGTTCATCTCCGAAATCACTATTCGCAATGATAACCAGGA |
| | | GGTGCTGCCCCAGCGCATCACCCATGACGTGGGCATCAAGC |
| | | CACTGAACCCCGACGATTTTTGGAGGTGCACTTCAGGACTG |
| | | CCTAGCCTGATGAAGACCCCAAAAATCCGACTGATGCCAGG |
| | | ACCTGGACTGCTGGCAATGCCAACCACAGTGGATGGATGCG |
| | | TCCGAACCCCCTCTCTGGTCATCAACGACCTGATCTACGCCT |
| | | ATACTAGTAATCTGATCACCCGCGGCTGTCAGGACATTGGG |
| | | AAGTCCTACCAGGTGCTGCAGATCGGCATCATTACAGTGAA |
| | | CAGTGATCTGGTCCCCGACCTGAATCCTCGCATCTCACACA |
| | | CTTTTAATATCAACGATAACCGAAAGTCATGCAGCCTGGCT |
| | | CTGCTGAACACAGACGTGTACCAGCTGTGCTCTACTCCTAA |
| | | AGTCGATGAACGGAGTGACTATGCAAGCTCCGGCATCGAGG |
| | | ATATTGTGCTGGACATCGTCAATTACGATGGGTCCATTTCTA |
| | | CTACCAGATTCAAGAACAATAACATCAGCTTTGACCAGCCC |
| | | TACGCCGCTCTGTATCCATCCGTGGGACCAGGAATCTACTA |
| | | CAAGGGAAAAATCATTTTCCTGGGCTATGGCGGGCTGGAAC |
| | | ACCCTATCAACGAGAATGTGATTTGCAACACAACTGGCTGT |
| | | CCAGGGAAGACCCAGAGGGATTGCAATCAGGCCAGTCATTC |
| | | ACCCTGGTTTAGTGATCGGAGAATGGTGAACTCAATCATTG |
| | | TGGTCGACAAAGGGCTGAATAGCATCCCTAAGCTGAAAGTC |
| | | TGGACCATTTCAATGCGACAGAACTACTGGGGAAGCGAAG |
| | | GCCGGCTGCTGCTGCTGGGCAATAAGATCTACATCTACACT |
| | | CGGAGCACCTCCTGGCACTCCAAACTGCAGCTGGGGATCAT |
| | | TGACATCACCGATTATTCTGACATCCGGATTAAGTGGACAT |
| | | GGCACAACGTGCTGTCAAGACCCGGGAATAACGAGTGTCCT |
| | | TGGGGACATAGCTGCCCAGATGGGTGTATCACCGGAGTGTA |
| | | CACAGACGCTTATCCTCTGAACCCAACCGGCAGTATCGTGT |
| | | CTAGTGTCATTCTGGACTCTCAGAAAAGTAGAGTGAATCCC |
| | | GTCATCACATACAGCACCGCAACAGAAAGAGTGAACGAGC |
| | | TGGCCATTCTGAATAGGACTCTGAGCGCCGGATATACCACA |
| | | ACTTCCTGCATCACCCATTACAACAAGGGCTATTGTTTCCAC |
| | | ATCGTGGAAATTAACCATAAAAGCCTGAATACCTTCCAGCC |
| | | CATGCTGTTTAAGACAGAGATTCCTAAAAGTTGTTCAGGCA |
| | | GTGGGTCAGGACATCACCATCATCATCATCATCACCATCAT |
| | | CACCATTGA |

The DNA encoding the tFrSc6-HN was codon optimized, synthesized with a N-terminal Kozak sequence (GCCACCATGG) and cloned into the NheIl, NotI sites of the episomal mammalian expression vector pEB4.3 (Genbank: MG182339). The regulatory components of this vector include an upstream human CMV promoter with intron IA with downstream elements WPRE (Woodchuck hepatitis virus posttranscriptional regulatory element) and BGHpA (bovine growth hormone polyadenylation signal).

Constructs were transfected into HEK293T cells and cells stably maintaining the episomal construct were selected for by puromycin resistance. Stably transfected cells were grown in SFM4HEK293 medium (Hyclone). The supernatant obtained by centrifugation was concentrated 5 fold and dialysed against equilibration buffer (50 mM Na phosphate, 300 mM NaCl, pH 8.0) by tangential flow. Protein was purified by affinity chromatography, more specifically by Immobilized Metal Chromatography (IMAC) using Ni60 Superflow (Clontech) according the manufacturer's instructions. The protein was finally dialysed against 2× PBS.

Recombinant proteins were subjected to SDS-PAGE to confirm purity (FIG. 1).

### B. Formulation

The formulation consists of 0.4 μg tFrSc6-HN protein and adjuvant (hereinafter referred to as “TriAdj”) in 50 μL deliverable volume per mouse and 2 μg tFrSc6-HN protein and adjuvant TriAdj in 100 μL deliverable volume per cotton rat. The TriAdj, consisted of 10 μg Poly-I:C, 20 μg HDP IDR-1002 peptide and 10 μg polyphosphazene. The polyphosphazene was either PCEP, CPZ37 or CPZ39.

In the second mouse trial and first cotton rat trial, aluminum hydroxide (“alum”) was also evaluated; 30% v/v aluminum hydroxide (alum; Invivogen; cat. Vac-Alu-250) was formulated with 0.4 μg tFrSc6-HN protein for mice in a 50 μL deliverable volume and 2 μg tFrSc6-HN protein for cotton rats in a 100 μL deliverable volume.

C. Immune Response in Mice

### Mouse Trial No. 1

BALB/c mice (n=5 per group) were vaccinated twice intramuscularly at a three-week interval either with PBS (control) or with tFrSc6-HN protein formulated with TriAdj containing either PCEP, CPZ37 or CPZ39. All groups were challenged three weeks after the second vaccination with RSV strain A2 (5×10⁵ PFU) and euthanized four days later.

tFrSc6-HN-specific IgG1 and IgG2a titers were determined by ELISA on serum collected before challenge. ELISA titers are expressed as the reciprocal of the highest dilution resulting in a value of two standard deviations above the negative control serum. The lower limit of detection is a titer of 40.

A virus neutralization (VN) assay was conducted for RSV and PIV3 on the serum collected individually at necropsy. VN titers are expressed as the highest dilution of serum that resulted in <50% of cells displaying cytopathic effects.

An enzyme-linked immunospot (ELISPOT) assay was performed to evaluate the induction of tFrSc6-HN-induced IFN-γ and IL-5 secreting cells in splenocytes. Cytokine-secreting cell numbers were expressed as the difference in the number of spots between tFrSc6-HN-stimulated wells and medium-control wells.

Virus replication in the lungs sampled at necropsy was tested for RSV. Virus replication is expressed as PFU per gram of lung tissue.

### Mouse Trial No. 2

BALB/c mice (n=5 per group) were vaccinated twice intramuscularly at a three-week interval with PBS (negative control), with tFrSc6-HN protein formulated with TriAdj containing either PCEP, CPZ37 or CPZ39, or with tFrSc6-HN formulated with aluminum hydroxide. All groups were challenged six weeks after the second vaccination with RSV strain A2 (5×10⁵ PFU). All animals were euthanized four days later.

tFrSc6-HN-specific IgG1 and IgG2a titers and RSV and PIV3 neutralization titers were determined as described for Mouse Trial 1. Virus replication in the lungs sampled at necropsy was tested for RSV as described for Mouse Trial 1.

A cytokine multiplex assay was used to determine the amounts of IFN-γ and IL-5 in all of the mice. Lung cytokines were quantified in the cell-free supernatants of lung homogenates using the electrochemiluminescence (ECL) detection-based Meso-scale discovery (MSD) multiplex platform and Sector Imager 2400 (MSD, Gaithersburg, MD, USA) according to the manufacturer's instructions.

### Cotton Rat Trial No. 1

In this trial, tFrSc6-HN was combined with different adjuvants: a triple adjuvant formulation (TriAdj) that contained polyI:C, IDR-1002 peptide and a polyphosphazene that was either PCEP, CPZ37, PCEP in a different formulation method, or aluminum hydroxide (Alum). Groups of 6 cotton rats were immunized twice with a 4-week interval with the tFrSc6-HN formulations. Control groups were immunized with live RSV or PBS. All cotton rats were challenged with RSV strain A2 (1×10⁶ PFU) three weeks after the last immunization and euthanized four days later tFrSc6-HN-specific IgG titers and RSV and PIV3 neutralization titers were determined as described for Mouse Trial 1. Virus replication in the lungs and the nasal washes sampled at necropsy was tested for RSV as described for Trial 1.

### D. Statistical Analysis

All data were analyzed using GraphPad PRISM version 7 for Windows (GraphPad Software). Differences among all groups were examined using one-way ANOVA. If a significant difference was found among the groups, median ranks between pairs of groups were compared by using the Mann-Whitney test. Differences were considered significant if P<0.05.

### 4. EXAMPLES

### A. Example 1: Immune Responses and Protection Generated by Administration of RSV-PIV3 Antigens in Mice

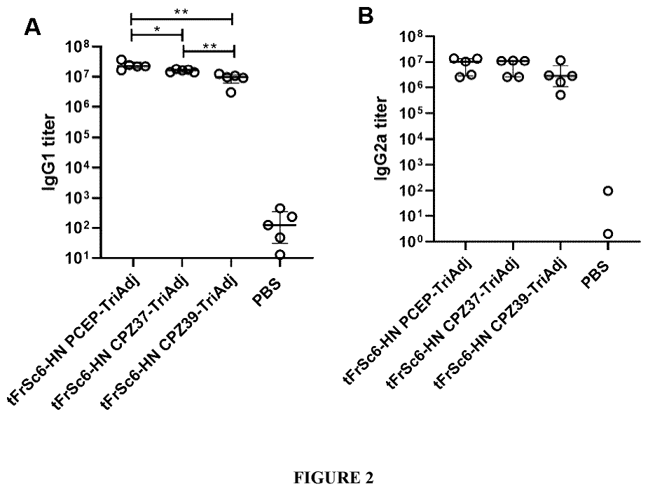

The ability of the RSV-PIV3 antigen tFrSc6-HN to induce immunity and protection against viral challenge was evaluated in mice in mouse Trial No.1. tFrSc6-HN was combined with triple adjuvant formulations differing in their polyphosphazene components: PCEP, CPZ37 or CPZ39. Groups of 5 mice were vaccinated twice with a 4-week interval, and challenged with RSV 3 weeks later. An additional group received PBS. All of the adjuvanted vaccines were able to induce significantly higher titers of IgG1 (FIG. 2A) and IgG2a (FIG. 2B) as compared to the PBS control, demonstrating immunogenicity. The group of mice vaccinated with tFrSc6-HN+CPZ39-TriAdj showed lower IgG1 titres than the two other TriAdj groups, tFrSc6-HN+PCEP-TriAdj and tFrSc6-HN+CPZ37-TriAdj, while tFrSc6-HN+PCEP-TriAdj elicited higher antibody responses than tFrSc6-HN+CPZ37-TriAdj. However, there were no significant differences between the three vaccinated groups in IgG2a titers.

The titers of IgG1 and IgG2a were similar, at approximately 107, for the tFrSc6-HN+PCEP-TriAdj and tFrSc6-HN+CPZ37-TriAdj vaccine groups, demonstrating a balanced TH2/TH1 response. While the tFrSc6-HN+CPZ39-TriAdj group developed lower antibody titers, the responses were also balanced.

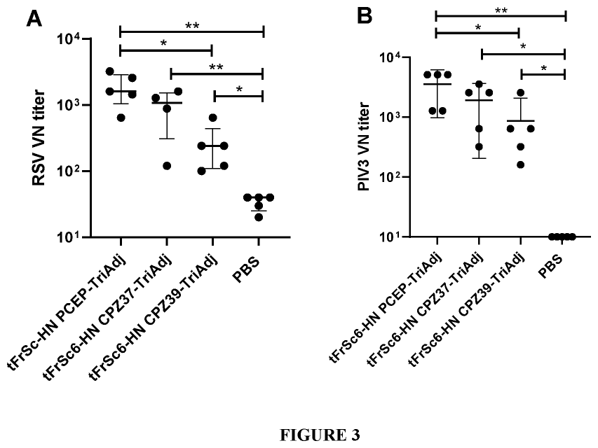

The serum antibodies generated by the three adjuvanted vaccines were shown to neutralize both RSV and PIV3 in a virus neutralization (VN) assay, with significantly higher titers induced in these groups than the serum antibodies induced by PBS (FIG. 3). Within the three adjuvanted vaccines, tFrSc6-HN+CPZ39-TriAdj induced lower RSV- and PIV3-neutralizing antibody titers than tFrSc6-HN+PCEP-TriAdj, while the RSV- and PIV3-VN titers in the tFrSc6-HN+CPZ39-TriAdj group tended to be lower than those of the tFrSc6-HN+CPZ37-TriAdj group. However, tFrSc6-HN+PCEP-TriAdj and tFrSc6-HN+CPZ37-TriAdj elicited similar VN antibody levels (FIG. 3A and B).

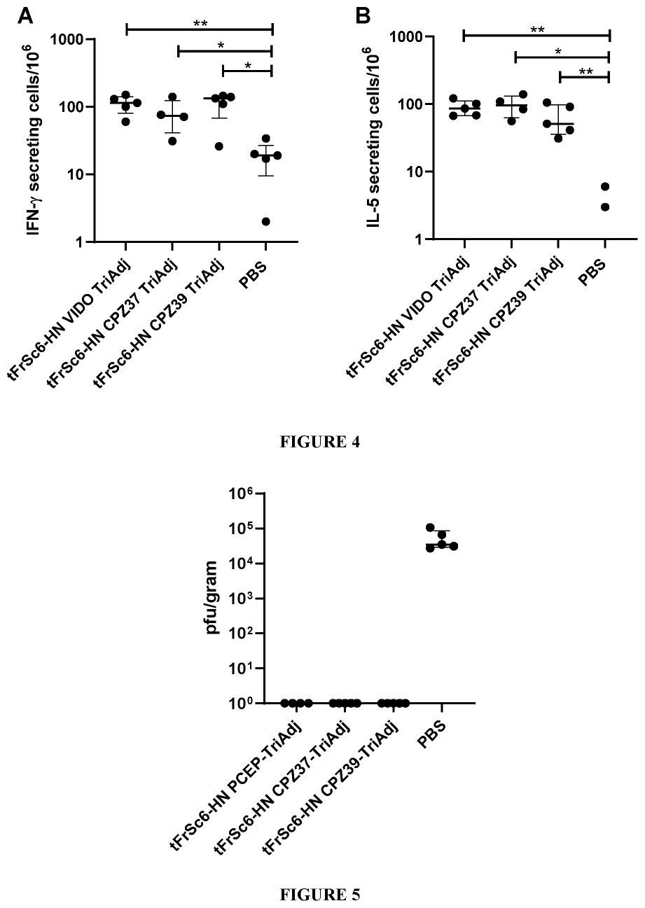

The three adjuvanted vaccine formulations induced tFrSc-HN-stimulated production of both IFN-γ and IL-5 secreting T cells, further supporting the induction of a balanced immune response (FIG. 4). There were no differences between the three adjuvant formulations.

After challenge with the RSV A2 strain, the amount of virus present in the lungs of all mice was determined. The three adjuvanted vaccines, tFrSc6-HN+PCEP-TriAdj, tFrSc6-HN+CPZ37-TriAdj and tFrSc6-HN+CPZ39-TriAdj, elicited complete protection, as observed by the absence of virus in any of the lungs of the three groups, while the lungs of mice vaccinated with PBS showed high levels of virus (FIG. 5).

### B. Example 2: Immune Responses and Protection Generated by Administration of RSV-PIV3 Antigens in Mice

The ability of the RSV-PIV3 antigen tFrSc6-HN to induce immune responses was evaluated in mouse Trial No.2. In this trial, tFrSc6-HN was combined with different adjuvants: the triple adjuvant formulation (TriAdj) that contained polyI:C, IDR-1002 peptide and a polyphosphazene that was either PCEP, CPZ37, or CPZ39; or aluminum hydroxide.

Groups of five mice were immunized twice with one of these vaccine formulations. One group of mice was immunized with PBS as a control. All mice were challenged six weeks later with RSV A2 and euthanized four days after challenge.

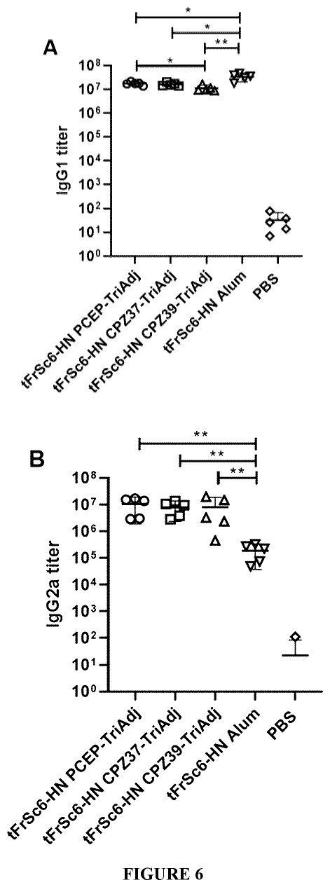

All groups immunized with a tFrSc6-HN-containing vaccine showed higher levels of IgG1 and IgG2a than the group vaccinated with PBS (FIG. 6A and 6B). The IgGI titer of the group immunized with tFrSc6-HN+CPZ39-TriAdj was lower than that of the animals that received tFrSc6-HN+PCEP-TriAdj, but there was no difference between the tFrSc6-HN+PCEP-TriAdj and the tFrSc6-HN+CPZ37-TriAdj groups. The tFrSc6-HN+Alum group induced higher IgG1 and lower IgG2a production than tFrSc6-HN formulated with any of the other adjuvants.

The three groups vaccinated with a vaccine that contained TriAdj (PCEP-TriAdj, CPZ37-TriAdj and CPZ39-TriAdj) developed a balanced TH1/TH2 immune response, as observed by IgG2a/lgG1 ratios close to 1. The alum-adjuvanted vaccine induced a typical TH2 response, with higher IgG1 levels (above 10⁷) than IgG2a levels (approximately 10⁵).

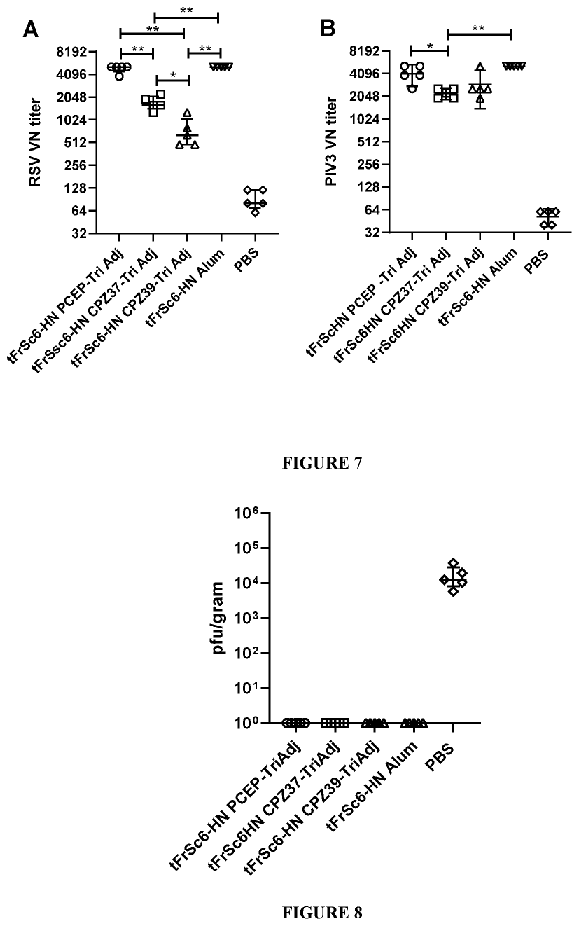

Furthermore, all groups of vaccinated mice developed VN titers against RSV and PIV3 (FIG. 7A and 7B). Within the vaccinated groups, the PCEP-TriAdj and alum groups developed higher SV-neutralizing titers than the CPZ37-TriAdj and CPZ39-TriAdj groups, and CPZ37 mediated induction of higher RSV-neutralizing titers than CPZ39. With respect to neutralization of PIV3, the PCEP-TriAdj and alum groups had higher titers than the CPZ37-TriAdj group, and tended to have higher titers than the CPZ39-TriAdj group.

The four adjuvanted vaccines, tFrSc6-HN+PCEP-TriAdj, tFrSc6-HN+CPZ37-TriAdj, tFrSc6-HN+CPZ39-TriAdj, and tFrSc6-HN+alum, elicited complete protection against RSV challenge, as observed by the absence of virus in any of the lungs of the three groups, while the lungs of mice vaccinated with PBS showed high levels of virus (FIG. 8).

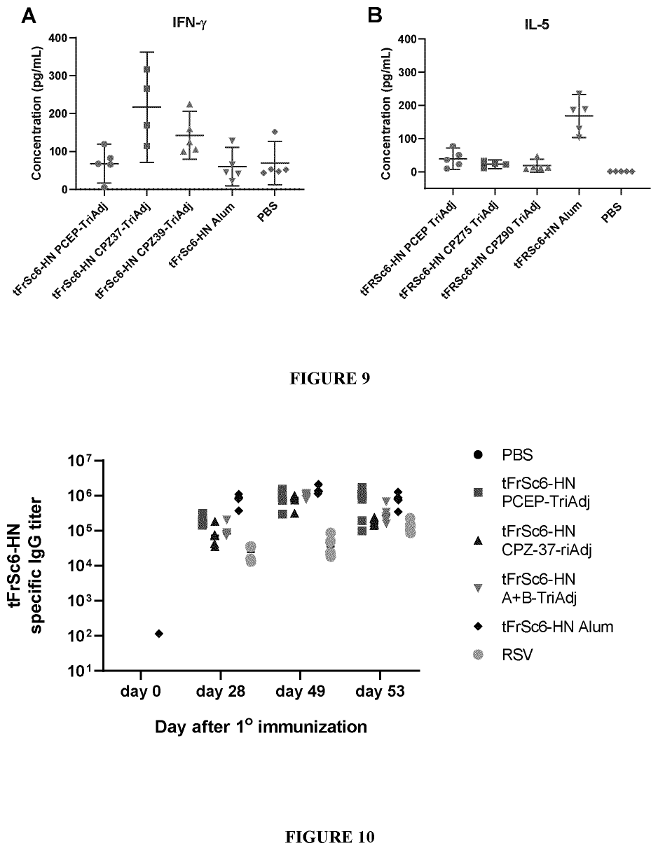

As a further measurement of the immune response bias, IFN-γ and IL-5 levels were measured in the lungs after RSV challenge. IL-5 was the predominant cytokine produced in the lungs of the alum-adjuvanted vaccine group, while the lungs of mice that were vaccinated with vaccine that contained TriAdj (PCEP-TriAdj, CPZ37-TriAdj and CPZ39-TriAdj) contained both IFN-γ and IL-5, with some predominance of IFN-γ in the CPZ37-TriAdj and CPZ39-TriAdj groups (FIG. 9), supporting the induction of a TH1 to balanced immune response.

### C. Example 3: Immune Responses and Protection Generated by Administration of RSV-PIV3 Antigens in Cotton Rats

The ability of tFrSc6-HN to induce protective immunity from RSV was further evaluated in cotton rats, which is an established model for RSV and PIV3. In this trial, tFrSc6-HN was combined with different adjuvants: TriAdj that contained polyI:C, IDR-1002 peptide and a polyphosphazene that was either PCEP, or CPZ37, or PCEP in a different formulation method (A+B), or aluminum hydroxide (Alum). Groups of 6 cotton rats were immunized twice with a 4-week interval with the tFrSc6-HN formulations. Control groups were immunized with live RSV or PBS. All cotton rats were challenged with RSV strain A2 three weeks after the last immunization and euthanized four days after challenge.

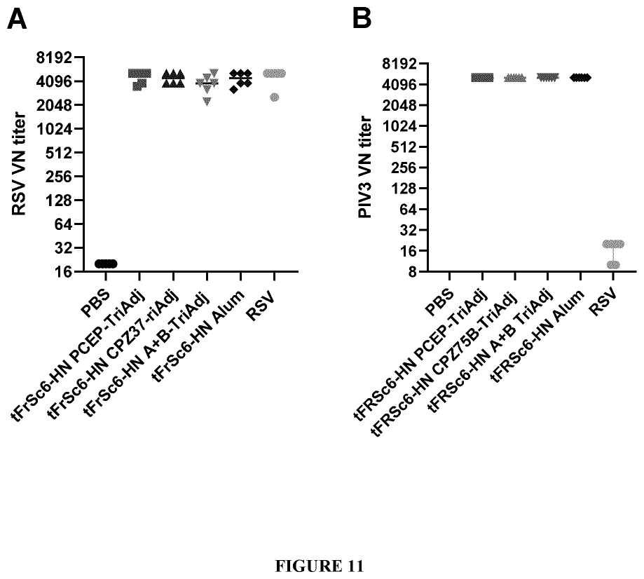

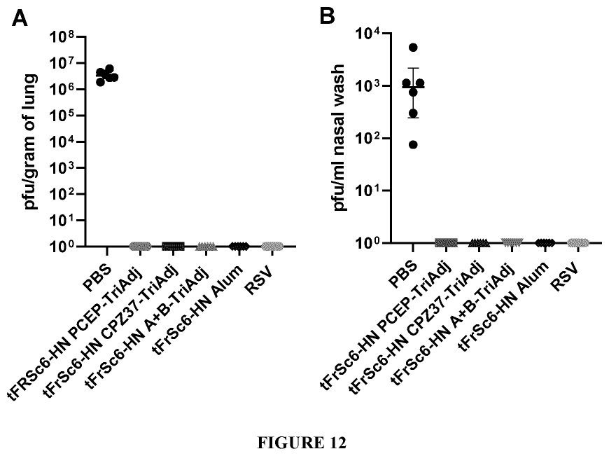

All cotton rats developed high tFrSc6-HN-specific serum IgG titers. The titers induced by live RSV were lower than those elicited by the RSV-PIV3 vaccine formulations on days 28 and 49 (FIG. 10), but this can be due to the antigen on the plates being tFrSc6-HN. There were no differences between the TriAdj-formulated tFrSc6-HN vaccine groups. Compared to the PBS group, the RSV- neutralizing antibody titers were higher in all vaccinated groups, and there were no differences between any of these groups (FIG. 11A). The PIV3-neutralizing antibody titers were high in all adjuvanted tFrSc6-HN-vaccinated groups, but as expected not in the RSV-vaccinated group. Accordingly, all vaccinated cotton rats were fully protected from RSV challenge; no virus was detected in the lungs or nasal washes (FIG. 12A and 12B).

The present invention has been described with regard to one or more embodiments. However, it will be apparent to persons skilled in the art that a number of variations and modifications can be made without departing from the scope of the invention as defined by the claims.

### REFERENCES

1) Ann R Falsey, Patricia A Hennessey, Maria A Formica, Christopher Cox, Edward E Walsh. Respiratory syncytial virus infection in elderly and high-risk adults. N Engl J Med 2005 Apr 28;352(17): 1749-59.
2) Shay, D. K., Holman, R. C., Roosevelt, G. E., Clarke, M. J. & Anderson, L. J. Bronchiolitis-associated mortality and estimates of respiratory syncytial virus-associated deaths among US children, 1979-1997. J Infect Dis 183, 16-22 (2001).
3) Walsh, E. E., Peterson, D. R. & Falsey, A. R. Risk factors for severe respiratory syncytial virus infection in elderly persons. J Infect Dis 189, 233-238, doi: 10.1086/380907 (2004).
4) Vainionpaa R, Hyypia T. Biology of parainfluenza viruses. Clinical microbiology reviews 1994 Apr; 7(2):265-75.
5) Henrickson K J, Hoover S, Kehl K S, Hua W. National disease burden of respiratory viruses detected in children by polymerase chain reaction. The Pediatric infectious disease journal 2004 Jan;23(1 Suppl):S11-8.
6) Reed G, Jewett P H, Thompson J, Tollefson S, Wright P F. Epidemiology and clinical impact of parainfluenza virus infections in otherwise healthy infants and young children<5 years old. The Journal of infectious diseases 1997 Apr; 175(4):807-13.
7) Schmidt, A. C., Schaap-Nutt, A., et al., 2011. Progress in the development of human parainfluenza virus vaccines. Expert Rev. Respir. Med. 5 (4), 515-526.
8) Moscona, A., 2005. Entry of parainfluenza virus into cells as a target for interrupting childhood respiratory disease. J. Clin. Investig. 115 (7), 1688-1698.
9) Collins, P. L., Graham, B. S., 2008. Viral and host factors in human respiratory syncytial virus pathogenesis. J. Virol. 82 (5), 2040-2055.
10) Guvenel, A. K., Chiu, C., et al., 2014. Current concepts and progress in RSV vaccine development. Expert Rev. Vaccine 13 (3), 333-344.
11) Higgins, D., Trujillo, C., et al., 2016. Advances in RSV vaccine research and development -a global agenda. Vaccine 34 (26), 2870-2875.
12) Garg, R., Brownlie, R., Latimer, L., Gerdts, V., Potter, A. and van Drunen Littel-van den Hurk, S. Vaccination with a human parainfluenza virus type 3 chimeric FHN glycoprotein formulated with a combination adjuvant induces protective immunity. Vaccine. 2017 Dec 18;35(51): 7139-7146.
13) Brideau, R. J., Oien, N. L., Lehman, D. J., Homa, F. L., Wathen, M. W., 1993. Protection of cotton rats against human parainfluenza virus type 3 by vaccination with a chimeric FHN subunit glycoprotein. J Gen Virol 74 (Pt 3), 471-477.
14) Healy, C. M., 2012. Vaccines in pregnant women and research initiatives. Clin. Obstet. Gynecol. 55, 474-486.
15) Stensballe, L. G., Ravn, H., Kristensen, K., Agerskov, K., Meakins, T., Aaby, P., Simoes, E. A., 2009. Respiratory syncytial virus neutralizing antibodies in cord blood, respiratory syncytial virus hospitalization, and recurrent wheeze. J. Allergy Clin. Immunol. 123, 398-403.
16) Halperin et al., A Randomized Controlled Trial of the Safety and Immunogenicity of Tetanus, Diphtheria, and Acellular Pertussis Vaccine Immunization During Pregnancy and Subsequent Infant Immune Response, Clinical Infectious Diseases, Volume 67, Issue 7, 1 October 2018, Pages 1063-1071.
17) Howard, T. S., Hoffman, L. H., Stang, P. E. & Simoes, E. A. Respiratory syncytial virus pneumonia in the hospital setting: length of stay, charges, and mortality. J Pediatr 137, 227-232 (2000).
18) Kim, H. W. et al. Epidemiology of respiratory syncytial virus infection in Washington, D.C. I. Importance of the virus in different respiratory tract disease syndromes and temporal distribution of infection. Am J Epidemiol 98, 216-225 (1973).
19) Venkatesh, M. P. & Weisman, L. E. Prevention and treatment of respiratory syncytial virus infection in infants: an update. Expert review of vaccines 5, 261-268 (2006).
20) Sung, R. Y., Murray, H. G., Chan, R. C., Davies, D. P. & French, G. L. Seasonal patterns of respiratory syncytial virus infection in Hong Kong: a preliminary report. The Journal of infectious diseases 156, 527-528 (1987).
21) Murphy B R, Prince G A, Collins P L, Van Wyke Coelingh K, Olmsted R A, Spriggs M K, et al. Current approaches to the development of vaccines effective against parainfluenza and respiratory syncytial viruses. Virus research 1988 Aug; 11(1):1-15.
22) Mansi C. Pandya, Sean M. Callahan, Kyryll G. Savchenko and Christopher C. Stobart. A Contemporary View of Respiratory Syncytial Virus(RSV) Biology and Strain-Specific Differences. Pathogens 2019, 8(2), 67.

What is claimed is:

1. An immunogenic fusion protein comprising a polypeptide with at least 90% sequence identity to a polypeptide selected from the group consisting of:
a polypeptide comprising amino acid residues 26 to 979 of SEQ ID NO: 1,
a polypeptide comprising amino acid residues 26 to 979 of SEQ ID NO: 3, and
a polypeptide comprising amino acid residues 26 to 979 of SEQ ID NO: 5.

2. An immunogenic composition comprising:
a polypeptide with at least 90% sequence identity to a fusion polypeptide selected from the group consisting of:
a polypeptide comprising amino acid residues 26 to 979 of SEQ ID NO: 1,
a polypeptide comprising amino acid residues 26 to 979 of SEQ ID NO: 3, and
a polypeptide comprising amino acid residues 26 to 979 of SEQ ID NO: 5,
and a pharmaceutically acceptable excipient.

3. The immunogenic composition of claim 2, wherein the composition further comprises an immunological adjuvant.

4. The immunogenic composition of claim 3, wherein the adjuvant comprises: (a) a polyphosphazene; (b) a CpG oligonucleotide or a poly (I:C); and (c) a host defense peptide.

5. The immunogenic composition of claim 1, wherein the composition is for administration to a human subject.

6. The immunogenic composition of claim 4, wherein the adjuvant is formulated with a mucoadhesive lipidic carrier to produce a mucoadhesive lipidic carrier system.

7. The immunogenic composition of claim 6, wherein the mucoadhesive lipidic carrier of the system comprises a cationic liposome.

8. The immunogenic composition of claim 7, wherein the mucoadhesive lipid carrier comprises one or more cationic lipids selected from 1,2-dioleoyl-3-trimethylammonium-propane (DOTAP); 3β-[N-(N′,N′-dimethylaminoethane)-carbamoyl] (DC); dimethyldioctadecylammonium (DDA); octadecylamine (SA); dimethyldioctadecylammonium bromide (DDAB); 1,2-dioleoyl-sn-glycerol-3-phosphoethanolamine (DOPE); egg L-α-phosphatidylcholine (EPC); cholesterol (Chol); distearoylphosphatidylcholine (DSPC); 1,2-dimyristoyl-3-trimethylammonium-propane (DMTAP); dimyristoylphosphatidylcholine (DMPC); or ceramide carbamoyl-spermine (CCS).

9. A method of treating or preventing RSV and/or PIV3 infection in a subject, comprising administering the fusion protein of claim 1 to said subject, such that said RSV and/or PIV3 infection is treated or prevented in said subject.

10. A method of treating or preventing RSV and/or PIV3 infection in a subject, comprising administering the immunogenic composition of claim 2 to said subject, such that said RSV and/or PIV3 infection is treated or prevented in said subject.

11. The method of claim 9 or 10, wherein the immunogenic composition further comprises an immunological adjuvant.

12. The method of claim 11, wherein the adjuvant comprises: (a) a polyphosphazene; (b) a CpG oligonucleotide or a poly (I:C); and (c) a host defense peptide.

13. The method of claim 9 or 10, wherein the subject is a human subject.

14. The method of claim 12, wherein the adjuvant is formulated with a mucoadhesive lipidic carrier to produce a mucoadhesive lipidic carrier system.

15. The immunogenic composition of claim 14, wherein the mucoadhesive lipidic carrier of the system comprises a cationic liposome.

16. The immunogenic composition of claim 15, wherein the mucoadhesive lipid carrier comprises one or more cationic lipids selected from 1,2-dioleoyl-3-trimethylammonium-propane (DOTAP); 3β-[N-(N′,N′-dimethylaminoethane)-carbamoyl] (DC); dimethyldioctadecylammonium (DDA); octadecylamine (SA); dimethyldioctadecylammonium bromide (DDAB); 1,2-dioleoyl-sn-glycerol-3-phosphoethanolamine (DOPE); egg L-α-phosphatidylcholine (EPC); cholesterol (Chol); distearoylphosphatidylcholine (DSPC); 1,2-dimyristoyl-3-trimethylammonium-propane (DMTAP); dimyristoylphosphatidylcholine (DMPC); or ceramide carbamoyl-spermine (CCS).

17. The method of claim 9 or 10, wherein the composition is administered parenterally, intramuscularly, intravenously, intraperitoneally, subcutaneously, orally, intranasally, or as an aerosol.
